(19) United States
(12) Patent Application Publication
(10) Pub. No.: US 2006/0078890 A1
(43) Pub. Date: Apr. 13, 2006

(54) Methods for identifying parkinson's disease therapeutics

(76) Inventor: Ole Isacson, Cambridge, MA (US)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/962,080

(22) Filed: Oct. 8, 2004

(51) Int. Cl. C12Q 1/68

(52) U.S. Cl. 435/6

(57) ABSTRACT

The present invention features methods of identifying candidate compounds for treating and preventing neurodegenerative disorders such as Parkinson's disease.

### FIELD OF THE INVENTION

The field of the invention relates to the treatment of neurodegenerative diseases.

### BACKGROUND OF THE INVENTION

Parkinson's disease (PD) is a progressive neurodegenerative disease characterized clinically by bradykinesia, rigidity, and resting tremor. The motor abnormalities are associated with a specific loss of dopaminergic neurons in the substantia nigra pars compacta (SNc) and depletion of striatal dopamine (DA) levels. While the loss of striatal DA correlates with the severity of clinical disability, clinical manifestations of PD are not apparent until about 80-85% of SNc neurons have degenerated and striatal DA levels are depleted by about 60-80%. DA neurons in the ventral midbrain consist of two main groups: the A9 group in the SN, and the A10 group in the medial and ventral tegmentum. Each of these cell groups projects to different anatomical structures and is involved in distinct functions. A9 cells mainly project to the dorsolateral striatum, involved in the control of motor functions, whereas A10 cells provide connections to the ventromedial striatum, limbic and cortical regions, involved in reward and emotional behavior. In addition to the distinct axonal projections and differences in synaptic connectivity, these groups of DA cells exhibit differences in neurochemistry and electrophysiological properties, illustrating functional differences despite similar neurotransmitter identity. These differences in A9 and A10 cells are also reflected in their specific responses to neurodegeneration in PD. Postmortem analyses in human PD brains demonstrate a selective cell loss of the A9 group with a survival rate of about 10% whereas the A10 group is largely spared with a survival rate of about 60%. This indicates that A9 cells are more vulnerable to intrinsic and/or extrinsic factors causing degeneration in PD. In addition, three regional gradients of neurodegeneration in the dorso-ventral/rostro-caudal/medio-lateral axis have been reported in PD. Caudally and laterally located ventral DA cells within A9 subgroups are the most vulnerable cells in PD. In contrast, the medial and rostral part of DA cell subgroups within A10 cells (i.e. rostral, linear nucleus, RLi) are the least affected (5-25% cell loss).

Currently, little is known about the mechanism underlying the neurodegenerative process and the basis for its differential effects on the A9 versus the A10 dopaminergic neurons. Accordingly, disease management is largely limited to strategies that achieve symptomatic relief (e.g., by replenishing dopamine levels) rather than strategies that seek to prevent or delay neurodegeneration. Thus, better treatment methods are needed for treating and preventing neurodegenerative disorders.

### SUMMARY OF THE INVENTION

The present invention features methods of identifying compounds useful for the treatment and prevention of Parkinson's disease (PD). The invention is based on our discovery of numerous genes that are differentially expressed in A9 dopaminergic neurons, which undergo a disproportionately high level of cell death in PD, compared to A10 dopaminergic neurons, which are relatively spared. Compounds that reduce or prevent neurodegeneration caused by PD can be identified using screening methods that employ the genes and/or polypeptides that are differentially expressed in neurodegeneration-sensitive (A9) and neurodegeneration-resistant (A10) cells. Screening methods that make use of a plurality of such genes and polypeptides allow for the identification of agents associated with an improved ability to specifically and effectively treat and prevent neurodegeneration.

In one aspect, the invention features a method of identifying a compound that treats or prevents Parkinson's disease in a human, involving the steps of: (a) providing cells that express at least five genes selected from Table 4; (b) contacting the cells with a candidate compound; and (c) assessing the expression level of the genes relative to the expression level of the genes in the absence of said candidate compound, wherein a candidate compound that reduces the expression of at least three of the genes is identified as a compound useful for treating or preventing Parkinson's disease.

In another aspect, the invention features a method of identifying a compound that treats or prevents Parkinson's disease in a human, involving the steps of: (a) providing cells that express at least five genes selected from Table 5; (b) contacting the cells with a candidate compound; and (c) assessing the expression level of the genes relative to the expression level of the genes in the absence of the candidate compound, wherein a candidate compound that increases the expression of at least three of the genes is identified as a compound useful for treating or preventing Parkinson's disease.

In particularly useful embodiments of these screening methods, at least 10, 20, 30, 50, 70, 100, 125, 150, or 200 genes are expressed by the cells and assessed for the level of expression.

In either of the foregoing screening methods, the genes selected from Table 4 and/or 5 may be expressed in a single cell type or in multiple cell types. For assays in which the selected genes are expressed in multiple cell types, the cell types may be mixed in a single heterogeneous culture or they may be segregated from the other cell types (i.e., a homogenous culture). Optionally, some or all of the cells may recombinantly express one or more of the selected genes. Thus, one test system might employ one cell line that naturally expresses one of the selected genes, one cell line transfected with two of the selected genes, and two additional cell lines, each of which is transfected with one of the selected genes. Many other variations of the system are possible such as using five or more cell lines, each of which is transfected with one of the selected genes.

Any mammalian cell type may be used in the foregoing methods. Human cells, rodent (e.g., rat and mouse) cells, and non-human primate cells are particularly useful. The cells may be cultured primary cells (e.g., cultured embryonic ventral mesencephalon cells) or immortalized cells. Useful immortalized cells include, for example, PC12 cells. Desirably, the PC12 cells recombinantly express α-synuclein.

Optionally, the cells may be further treated with a compound that induces cell death (e.g., necrotic death or apoptosis). Such compounds are desirably mitochondrial complex I inhibitors including, for example, 1-methyl-4-phenylpyridinium (MPP⁺), rotenone, isoquinoline, tetrahydroisoquinoline, or 6-hydroxydopamine.

Any method for assessing gene expression is useful in the foregoing methods. For example, the level of gene expression may be assessed by measuring the RNA levels transcribed from the selected genes using techniques such as Northern blotting and/or RT-PCR. Alternatively, the amount of protein expressed by the selected genes may be measured, for example, by Western blotting, ELISA, or measuring the biological activity of protein.

The invention also provides a method of identifying a compound for treating or preventing Parkinson's disease involving the steps of: (a) providing a cell having a reporter gene operably linked to the promoter of a gene selected from Table 4; (b) contacting the cell with a candidate compound; and (c) assessing the level of expression of the reporter gene relative to the level of expression of the reporter gene in the absence of the candidate compound, wherein a candidate compound that reduces the level of expression of the reporter gene is identified as a compound that is useful for the treatment or prevention of Parkinson's disease.

The invention also provides a method of identifying a compound for treating or preventing Parkinson's disease involving the steps of: (a) providing a cell having a reporter gene operably linked to the promoter of a gene selected from Table 5; (b) contacting the cell with a candidate compound; and (c) assessing the level of expression of the reporter gene relative to the level of expression of the reporter gene in the absence of the candidate compound, wherein a candidate compound that increases the level of expression of the reporter gene is identified as a compound that is useful for the treatment or prevention of Parkinson's disease.

Any suitable reporter gene may be used in either of the two foregoing methods. Particularly useful reporter genes include, for example, glucuronidase (GUS), luciferase, chloramphenicol transacetylase (CAT), green fluorescent protein (GFP), alkaline phosphatase, and β-galactosidase. Optionally, the cells may be further contacted with a mitochondrial complex I inhibitor.

Any mammalian cell type is suitable for use in these reporter gene assays. Human cells, rodent (e.g., rat and mouse) cells, and non-human primate cells are particularly useful. The cells may be immortalized cells or they may derived from cultured primary cells (e.g., cultured embryonic ventral mesencephalon cells). Useful immortalized cells include, for example, PC12 cells. Desirably, the PC12 cells also recombinantly express α-synuclein.

The invention also features a solid support surface (e.g., multiwell plate or slide) containing 1000 or fewer unique polynucleotide probes capable of binding at least 200 distinct nucleic acids that encode genes contained in Table 4 and Table 5, wherein the probes are arranged on the surface such that, when contacted with a sample containing said nucleic acids, each binding event is segregated from the others.

In preferred embodiments, the polynucleotide probes are capable of binding at least 300 or at least 400 distinct nucleic acids that encode genes contained in Table 4 and Table 5. Desirably, the polynucleotide probes are conjugated to a detectable label such as a fluorescent label or an enzyme tag. Useful labels include, for example, digoxigenin, β-galactosidase, urease, alkaline phosphatase, peroxidase, or an avidinibiotin complex.

By a “promoter” is meant a nucleic acid sequence sufficient to direct transcription. Also included in the invention are those promoter elements which are sufficient to render promoter-dependent gene expression controllable for cell type-specific, tissue-specific or inducible by external signals or agents; such elements are usually located in the 5′ region of the native gene, but may also be found in the 3′ regions.

By “operably linked” is meant that a nucleic acid molecule and one or more regulatory sequences (e.g., a promoter) are connected in such a way as to permit expression of the gene product (i.e., RNA) when the appropriate molecules (e.g., transcriptional activator proteins) are bound to the regulatory sequences.

By a “mitochondrial complex I inhibitor” is meant any compound that reduces the activity of the mitochondrial respiratory chain complex thereby reducing the oxidative phosphorylation and/or increasing production of mitochondrial reactive oxygen species. The reactive oxygen species may in turn result in the induction of apoptosis. Exemplary mitochondrial complex I inhibitors are 1-methyl-4-phenylpyridinium (MPP⁺), rotenone, isoquinoline, and tetrahydroisoquinoline.

By “specifically binds,” when referring to an interaction between distinct molecules such as polynucleotides, is meant a binding event for which the target molecule and the receptor molecule (e.g., probe) interact with high affinity and specificity. The receptor molecule does not substantially bind to any other non-target molecules that are present. Preferably, the specific binding contributes 75%, 85%, 90%, 95%, 99%, or 100% of the total binding detected in the sample.

### BRIEF DESCRIPTION OF THE FIGURES

FIG. 1A is a tyrosine hydroxylase (TH)-immunostain of the coronal section of the mouse midbrain. A9 dopaminergic neurons are located in the substantia nigra pars lateralis (SNl) and the substantia nigra pars compacta (SNc). A10 dopaminergic neurons are located in the ventral tegmental area (VTA), the nucleus paranigralis (PN), and the interfascicular nucleus (IF).

FIGS. 1B-1D are TH-immunostains showing brain sections that were used in the laser capture microscopy (LCM) procedure. FIG. 1B shows TH-positive neurons in the SNc before laser capture. In FIG. 1C, TH-positive cells were targeted for laser capture using a 7.5 μm diameter-laser. Captured cells on the thermoplastic film were visualized before processing for RNA extraction (FIG. 1D).

FIG. 1E is a bar graph showing GIRK2 and calbindin mRNA levels in LCM samples, as measured by real time PCR. GIRK2 was increased by 2.78 fold (SEM±0.95) in A9 cells and calbindin was increased by 2.90 fold (SEM±0.64) in A10 cells.

FIGS. 1F-1H are bar graphs showing the functional profiles of microarray data. Genes that were induced in A9 and A10 cells were categorized based on their biological functions and cellular components by Onto-Express. Genes associated with a fold induction of greater than 1.5 (FDR<5%) were distributed into different categories, such as metabolism (FIG. 1F), cellular component in cytoplasm (FIG. 1G), transport, and signal transduction mechanism (FIG. 1H).

FIGS. 2A and 2B show a series of pictures of PC12-α-Synuclein (Syn)-overexpressing GIRK2 cells. Cells were infected with an eGFP or GIRK2-expressing lentivirus at an MOI of 5. Higher GIRK2 expression was achieved in GIRK2-infected cells relative to eGFP-infected control cells. Red staining represents GIRK2 and cell nuclei was visualized by DAPI staining (blue).

FIG. 2C is a bar graph showing cell death induced by MPP⁺ toxicity (1 and 5 mM) in PC12-αSyn cells, as measured by LDH release. LDH release was significantly increased in GIRK2-overexpressing cells compared to control eGFP-overexpressing cells (*; p<0.05 between groups).

FIG. 2D is a bar graph showing cell death induced by MPP⁺ toxicity in PC12-αLSyn, as measured by LDH release. LDH release was significantly reduced by FGF1 treatment (*; p<0.05 between groups). A Student's t-test was used to obtain statistical significance.

FIGS. 3A-3E are bar graphs showing cell death induced by MPP⁺ toxicity in PC12-αSyn. These graphs demonstrate the neuroprotective effects that results from increasing concentrations of NT-3 and several neuropeptides. Cells were pretreated with NT-3, VIP, CGRP, CCK-8, or GRP for two hours prior to 1 mM MPP⁺ treatment for 24 hours. The levels of LDH release were presented as a percent of control group without treatment. Significant dose-dependent reductions in LDH release were detected in these experiments indicating neuroprotective effects of these peptides from toxic insult (*; p<0.01 in the comparison with the group of MPP⁺ only treatment).

FIG. 4A is a series of immunostains of primary VM cells. Immunostaining of primary VM culture show TH-positive (left), GIRK2-positive (middle), and TH-positive/GIRK2-positive cells (right).

FIG. 4B is a bar graph representing the number of dopaminergic neurons as a at various MPP⁺ concentrations. TH-positive/GIRK2-positive or TH-positive/GIRK2-negative cells were stereologically counted following MPP⁺ treatment and were presented as % of total TH-positive neurons of control conditions. TH-positive/GIRK2-positive cells were more vulnerable to MPP⁺ than TH-positive/GIRK2-negative cells (*; p<0.05 in the comparison with the control condition).

FIG. 4C is a bar graph showing the number of GIRK2-positive dopaminergic neurons. GRP was added to a primary VM culture at day 5, two hours prior to treatment with MPP⁺ (10 μM). After 48 hours, TH-positive/GIRK2-positive neurons were stereologically counted as A9-like dopaminergic neurons and presented as % of total TH-positive/GIRK2-positive neurons of control (no MPP⁺) conditions (*; p<0.05 in the comparison with the group of MPP⁺ only treatment (black bar graph)). A Student's t-test was used to obtain statistical significance.

FIGS. 5A-5C are graphs showing differential gene expression between A9 and A10 cells. All genes were plotted on a log scale and represent a comparison between samples of A9- and A10-microdissected samples. Five A9 replicates are plotted against six A10 replicates to determine the differential gene expression between these groups. The distance from the mid-line reflects increasing levels of differential gene expression. All data were normalized using the probe level Robust Multi-Chip Analysis (RMA) algorithm.

FIGS. 6A-6D is series of photomicrographs of TH/GIRK2-immunostained E14 primary cell cultures. Scale bar in FIGS. 6A-6C=50 μm. Scale bar in FIGS. 6D=10 μm.

FIGS. 6E-6G are photomicrographs of immunostained E14 primary cell cultures. The effect of NT3 on survival of TH (green) and TH/GIRK2 (yellow) double-labeled neurons in primary VMDA culture is shown. NT3 at 17 ng/ml protects the cells against MPP⁺ toxicity (3 mM). Scale bar=100 μm.

FIGS. 7A-7C are photomicrographs of immunostained E14 primary cell cultures demonstrating the neuroprotective effect of VIP in primary VM culture. Immunostaining against TH (green) and GIRK2 (red) was performed 48 hours after 10 μM of MPP⁺ was applied to VM culture in the absence or presence of 10⁻⁶ M VIP.

FIG. 8 shows a series of graphs representing cell death in DAN cells, as measured by LDH release. MPP⁺ administration provides a toxic insult to DAN cells, which is maximal at a dose of 2.5 mM. NT-3 is neuroprotective since cells pretreated with NT-3 for two hours prior to MPP⁺ treatment (1 mM) show a significant dose-dependent reduction in LDH release.

FIGS. 9A-9I are photomicrographs of tyrosine hydroxylase (TH) immunohistochemistry of the ipsilateral striatum following intrastriatal 6-OHDA administration. The time course following 6-OHDA administration is: FIG. 9A=control; FIG. 9B=3 days; FIG. 9C=5 days; FIG. 9D=10 days; FIG. 9E=21 days; and FIG. 9F=28 days. Striatal TH fiber loss and nigral TH cell loss is seen in 6-OHDA lesioned rats. These immunostains show the conversion of the normal innervation of the contralateral side of the lesion to a fully denervated striatum 28 days after 6-OHDA striatal lesion. FIGS. 9G-9I are photomicrographs of tyrosine hydroxylase (TH) immunohistochemistry of the substania nigra 0, 10, and 28 days after intrastriatal 6-OHDA administration, respectively. There is a progressive loss of dopaminergic neurons on the lesioned side at 10 and 28 days compared to the unlesioned side.

FIGS. 10A and 10B are bar graphs quantifying the progressive loss of dopaminergic neurons in the substantia nigra of 6-OHDA lesion model rats. At post-lesion day 21, the continued loss of the TH positive cells can be prevented by delivering neurotrophic and neuroprotective molecules or genes.

FIG. 11 is a series of photomicrographs from experiments using eGFP lentiviral expression vector. PC12 and primary ventral mesencephalic dopaminergic (VMDA) neurons were transduced with an eGFP-expressing lentivirus at an MOI of 5 and 3, respectively. Four days after transduction, cells were stained with Rhodamine Red-labeled α-synuclein (PC12) or TH-specific (primary VMDA cell cultures) antibodies and analyzed using a confocal microscope (upper two rows). One hundred percent transduction efficiency was observed for PC12 cells and TH-expressing primary neurons. For in vivo transduction, SN of rats was injected with 10⁶ to 2×10⁶ TU of the lentivirus and eGFP expression was analyzed by fluorescence microscopy 6 days after injection. An exemplary virus-transduced SN stained with an anti-TH antibody is shown in the lower two rows. The majority of the midbrain TH-positive neurons were transduced with the eGFP-expressing lentivirus (yellow cells marked by arrows in high magnification).

### DETAILED DESCRIPTION

In order to understand the innate physiological differences between A9 and A10 neurons for the purpose of identifying novel therapies and therapeutic targets, and developing drug screening and diagnostic assays for Parkinson's disease (PD), individual A9 and A10 dopaminergic neurons were isolated by laser capture microdissection (LCM) and genomic profiles assessed by microarray analysis. The results show differential gene expression in A9 and A10 which may contribute to their altered vulnerability in PD. Further, differential expression of selected proteins in in vitro dopaminergic cells (e.g., α-synuclein-overexpressing PC12 cells (PC12-α-syn) and primary ventral mesencephalic (VM) cultures) alters the cells' sensitivity to MPP⁺ toxicity. Several of the polypeptides identified by microarray analysis were also differentially expressed within reported Parkinson's disease linkage intervals. Accordingly, proteins that are preferentially expressed in A10 cells (Table 5) may increase the cellular threshold to the neurodegenerative process in Parkinson's disease; whereas, proteins preferentially expressed in A9 cells (Table 4) may be responsible for their susceptibility to degeneration in Parkinson's disease.

Differential Expression Between A9 and A10 Dopaminergic Neurons

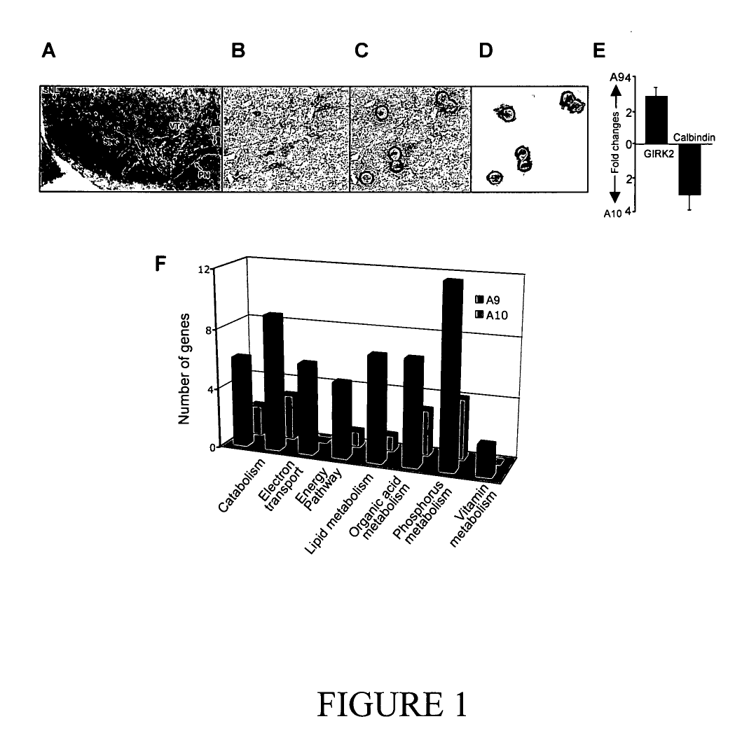

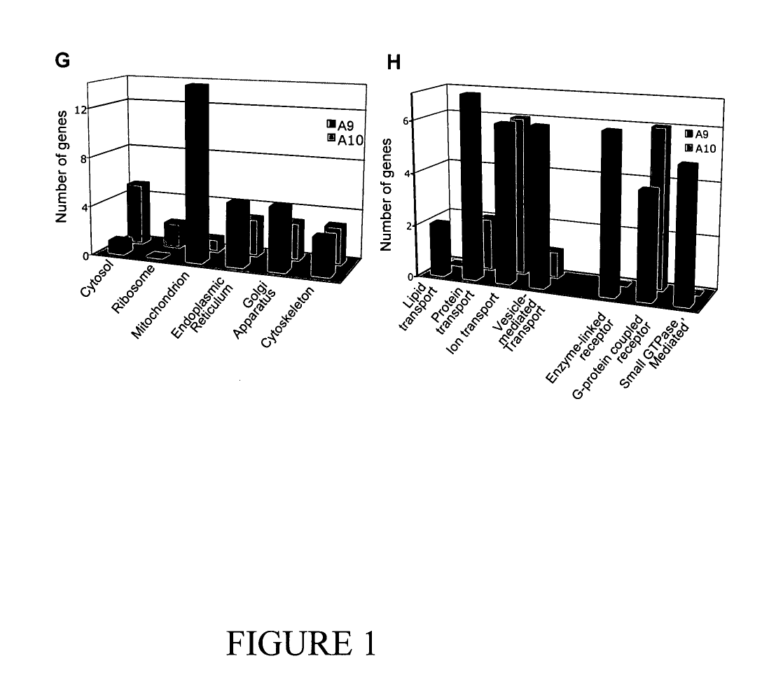

Midbrain A9 and A10 DA neuronal groups were identified by rapid TH-immunostaining to minimize RNA degradation in tissue sections and were next microdissected by laser-capture microscopy using anatomical criteria (FIGS. 1A-D). The quality of the extracted RNA samples was validated by real time PCR of G-protein coupled inward rectifying potassium channel isoform 2 (GIRK2), a molecule known to be more expressed in A9 DA neurons, and calbindin, a molecule known to be more expressed in A10 DA neurons (FIG. 1E).

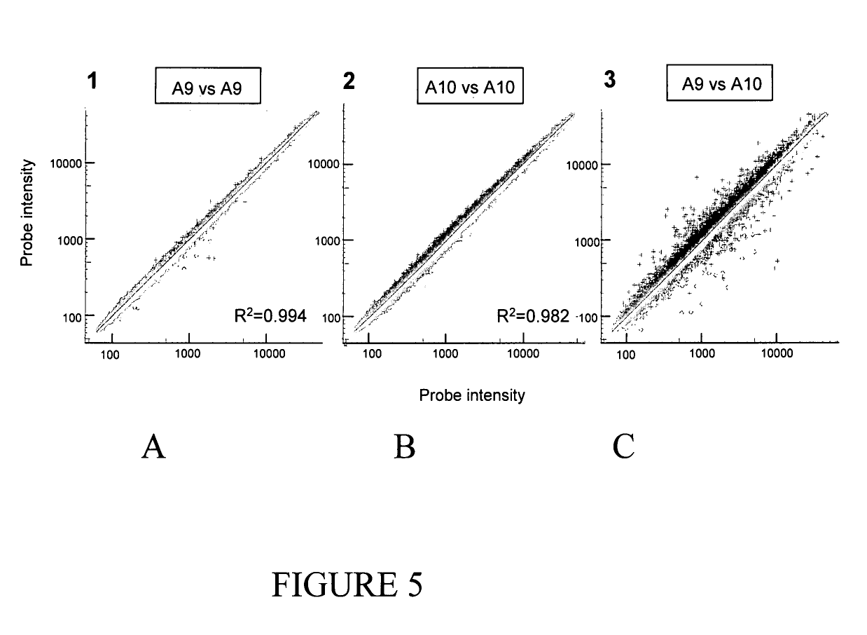

Microarray analysis was performed to investigate the molecular differences between dopaminergic neurons located in the A9 and A10 midbrain regions. Five biological replicates from A9 regions and six biological replicates from A10 regions of mouse brain were analyzed on an Affymetrix Murine 430A high-density oligonucleotide array, which currently queries 20,000 murine probe sets. Paired hybridization results between replicates of A9 (FIG. 5A) and A10 (FIG. 5B) samples demonstrated the reproducibility between the biological replicates. The distribution of gene expression signal from the combined data of the A9 and A10 series displayed the similarity and even distribution of up-and down-regulated genes in the plot (FIG. 5C). The differences that distinguished these two neuronal populations illustrated that only a small number of genes were differentially expressed. Forty-six genes had greater than 2.0-fold elevation of mRNA levels in A9 compared to A10 DA neurons, and 199 genes, greater than 1.5-fold (false discovery rate [FDR]<5%). Sixty-one genes had greater than 2.0-fold elevation of mRNA level in A10 compared to A9 DA neurons and 163 genes, greater than 1.5 fold (FDR<5%) (Tables 4 and 5).

The microarray analysis was validated in two ways. Firstly, previously reported gene expression differences between A9 and A10 DA neurons were verified. For example, Raldh1 was more expressed in A9, and calbindin D28K and cholecystokinin more in A10 DA neurons (Tables 4 and 5). Secondly, using real time PCR of laser-captured RNA samples, the mRNA levels of several genes from our microarray analysis was quantified to confirm A9/A10 gene expression patterns (Table 1). From various functional pathways, genes with relatively high mRNA expression and/or potential biological association to relative vulnerability were selected for validation.

TABLE 1
Relative Overexpression of Selected Genes in Midbrain DA Neurons

| | | | | Fold |
|---|---|---|---|---|
| | Category | Accession | Gene | change |
| A9 | Growth factor | NM_010197 | Fibroblast growth factor 1 | 4.7 ± 0.4 |
| | | NM_010512 | Insulin-like growth factor 1 | 2.5 ± 0.1 |
| | | NM_008010 | Fibroblast growth factor receptor 3 | 3.7 ± 1.3 |
| | Mitochondrial protein | NM_007451 | Adenine nucleotide translocator 2 | 2.4 ± 0.4 |
| | Energy metabolism | NM_008492 | Lactate dehydrogenase 2, B chain | 2.6 ± 0.5 |
| | Vesicle mediated | NM_026697 | RAB14 | 2.2 ± 0.2 |
| | transport | | | |
| | Enzyme-linked | NM_011219 | Protein tyrosine phosphatase | 2.3 ± 0.4 |
| | receptor/phosphatase | | z-polypeptide 1 | |
| | Apoptosis | NM_007611 | Caspase 7 | 2.6 ± 1.1 |
| | Cell surface | NM_009846 | CD24a | 5.6 ± 0.9 |
| | marker | | | |
| A10 | Neuropeptide | NM_013732 | Cocaine and amphetamine regulated | 4.7 ± 0.3 |
| | | | transcript | |
| | | NM_011702 | Vasoactive intestinal polypeptide | 4.4 ± 1.5 |
| | | NM_175012 | Gastrin releasing peptide | 8.5 ± 2.0 |
| | Calcium binding | NM_009037 | Reticulocalbin | 4.3 ± 0.1 |
| | protein | NM_009788 | Calbindin D28K | 2.9 ± 0.6 |
| | Lipid metabolism | NM_008509 | Lipoprotein lipase | 5.3 ± 1.7 |
| | Phosphatase inhibitor | NM_011153 | G-substrate | 2.9 ± 1.0 |

To gain insight into the biological relevance of differential A9/A10 gene expression, genes exhibiting differences greater than 1.5 fold (FDR<5%) were analyzed with Onto-Express (OE) software, which systematically translates genetic input into functional profiles. Genes from several categories showed striking differences between cell groups. Genes related to metabolism (FIG. 1F) and genes encoding mitochondrial proteins (FIG. 1G) were more expressed in A9 DA neurons compared to A10 DA neurons. Genes involved in protein, lipid and vesicle-mediated transport, but not ion transport, were also found to be more highly expressed in A9 DA neurons (FIG. 1H). In addition, among a number of genes involved in signal transduction mechanisms, enzyme-linked receptor genes such as receptor tyrosine kinases and receptor tyrosine phosphatases were more expressed in A9 DA neurons (FIG. 1H); whereas, all of the signal transduction genes with elevated expression in A10 fell into the category of G-protein coupled receptors. Notably, several genes related to small GTPase-mediated signaling and synaptic vesicle recycling, including RAB and RAS proteins, were more expressed in A9 DA neurons (FIG. 1H) and genes related to neuropeptide and hormone activity were typically elevated in A10 cells (Table 2).

TABLE 2
Differential Expression of Selected Genes in Neuronal Subpopulations

| | A9 | A10 |
|---|---|---|
| Neuropeptide/ | Secreted phosphoprotein 1 | Cocaine and amphetamine regulated |
| hormone | Neueopeptide Y receptor Y5 | transcript |
| activity | Neurotensin receptor 2 | Calcitonin/calcitonin-related |
| | | polypeptide, alpha |
| | | Colony stimulating factor 2 receptor, |
| | | beta 2 |
| | | Growth hormone receptor |
| | | Gastrin releasing peptide |
| | | Vasoactive intestinal polypeptide |
| | | Tachykinin receptor 3 |
| | | Adenylate cyclase activating |
| | | polypeptide 1 |
| | | Cholecystokinin |
| | | Arginine vasopressin receptor 1A |
| Growth factor | Fibroblast growth factor 1 | |
| activity | Fibroblast growth factor receptor 3 | |
| | Fibroblast growth factor inducible 15 | |
| | Insulin-like growth factor 1 | |
| | Epidermal growth factor pathway | |
| | substrate 8 | |
| Protease | Caspase 7 | |
| | Protease, serine, 11 (Igf binding) | |
| Protease | | Plexin C1 |
| inhibitor | | Cysteine-rich motor neuron 1 |
| | | Serine protease inhibitor, Kunitz |
| | | type2 |
| Phosphatase | Protein tyrosine phosphatase z | |
| | polypeptide 1 | |
| | Protein phosphatase 2A, regulatory | |
| | subunit B (B56) | |
| Phosphatase | | G-substrate |
| inhibitor | | |
| Apoptosis | Caspase 7 | Pleomorphic adenoma gene-like 1 |
| | Bcl-2 like 11 (apoptosis facilitator) | Mitogen activated protein kinase |
| | Estrogen-related receptor β like 1 | kinase kinase 5 |
| | Programmed cell death 10 | |

Certain opposing molecular functional categories exhibit inverse expression patterns in A9 and A10 DA neurons. For example, gene expression of proteases and phosphatases are elevated in A9 DA neurons; whereas, inhibitors of proteases and phosphatases were more highly expressed in A10 DA neurons (Table 2). Two pro-apoptotic genes, caspase 7 and Bcl2-like 11, are more highly expressed in A9 cells (Table 2).

The microarray data was further analyzed by comparing the genomic profiles with candidate susceptibility genes for PD generated by previous genomic convergence analysis that combined a genomic linkage and expression analysis (Hauser et al., Hum. Mol. Genet. 12: 671-7, 2003). That study reported 402 genes in the human substantia nigra that lay within five large genomic linkage regions identified in 174 PD families. Several differentially expressed genes in A9 and A10 DA neurons from the present microarray analysis were identical or similar to genes within the reported PD linkage intervals (Table 3).

TABLE 3
Concurrence Between Human Genomic Convergence Analysis in PD Families
and Murine Microarray Analysis

| | | Differentially expressed genes in A9 and A10 | | |
|---|---|---|---|---|
| Gene located in PD linkage interval from | | from Mouse Microarray Analysis | | |
| Human Genomic Convergence Analysis | | | | A9/ |
| Unigene | Description | Unigene | Description | A10 |
| Hs.75297 | FGF1 | Mm.5087 | FGF1 | A9 |
| Hs.7688 | PP2A regulatory subunit B | Mm.3785 | PP2A regulatory subunit B | A9 |
| | Beta isoform | | Gamma isoform | |
| | | Mm.46735 | RAB3C | A9 |
| | | Mm.1387 | RAB11a | A9 |
| | | (Mm.14530) | RAB1 | A9 |
| | | Mm.271944 | | |
| Hs.5807 | RAB14 | Mm.198264 | RAB14 | A9 |
| Hs.301853 | RAB34 | | | |
| Hs.479 | RAB5C | | | |
| Hs.55099 | RAB6 GTPase activating protein | | | |
| | (GAP and centrosome associated) | | | |
| Hs.19012 | RAB9 effector p40 | | | |
| Hs.31547 | NADH dehydorgenase | Mm.44227 | NADH dehydrogenase | A9 |
| | (ubiquinone) 1 alpha subcomplex, 8 | | (ubiquinone) Fe—S protein 8 | |
| Hs.79172 | Solute carrier family 25 | Mm.658 | Solute carrier family 25 | A9 |
| | (mitochondrial carrier; adenine | | (mitochondrial carrier; adenine | |
| | nucleotide translocator), member 5 | | nucleotide translocator), member 5 | |
| Hs.8262 | Lysosomal-associated membrane | Mm.197518 | Lysosomal-associated membrane | A9 |
| | protein 2 | | protein 4B | |
| Hs.90998 | Septin6 | Mm.2214 | Septin4 | A9 |
| Hs.37144 | MAGUK p55 subfamily member 3 | Mm.20449 | MAGUK p55 subfamily member 3 | A10 |
| Hs.3972 | Sialyltransferase 7D | Mm.4954 | Sialyltransferase 8B | A10 |
| Hs.82302 | Heparin sulfate 6-sulfotransferase 2 | Mm.41264 | Heparin sulfate 6-sulfotransferase 2 | A10 |

In Vitro Models of Parkinson's Disease

In vitro models, using a wide array of cell types, may be used in the screening methods of this invention to identify candidate compounds for the treatment of PD. These models also may be used to tested the effects of the candidate compounds on cell survival and neurodegeneration. Primary fetal dopaminergic neurons or cell lines exhibiting some characteristics of the dopaminergic neuronal phenotype may be used in the present invention. Cell lines have the advantage of providing a homogeneous cell population, which allows for reproducibility and sufficient number of cells for experiments. Primary dopaminergic cultures are derived from tissues harvested from developing ventral mesencephalon (VM) containing the substantia nigra. They have the advantage of containing authentic dopaminergic neurons cultured in a context of their naturally occurring neighboring cells. In our studies, we also employed human dopaminergic neuron progenitor (DAN) cells, using an isolation technique that produces up to 70% dopaminergic neurons with robust immunoreactivity for dopamine, tyrosine hydroxylase (TH), and dopamine transporter (DAT) following in vitro differentiation. We also employed cell culture models characterized by abnormal protein aggregation and degradation, which are related to the ax-synuclein/ubiquitin-proteasome system (UPS). In these models, the overexpression of wild type α-synuclein causes an impaired mitochondrial respiratory chain function. The overexpression of mutant α-synuclein (A53T and A30P) produces oxidative stress and an impaired function of the UPS, leading to apoptosis (A53T). The inducible expression of mutant α-synuclein (A30P) in PC12 cells reduces proteasome functions and increases mitochondria-dependent apoptosis after sub-threshold toxic concentrations of proteasome inhibitor (lactacystin). Any of these cell culture models are useful for the screening methods of the invention and are described in more detail below.

Tet-Inducible (Tet-Off) α-Synuclein Overexpressing PC12 Cells

A PC12 cell line was transfected to express wild type human α-synuclein using the Tet-Off system (Clontech Laboratories, Palo Alto, Calif.). α-Synuclein expression was suppressed using 1 μg/mL doxycycline (Clontech) in culture media before expression is required. α-Synuclein expressing PC12 cells (PC12-αSyn) were grown in Dulbecco's modified Eagle's medium (DMEM) (Invitrogen, Calsbad, Calif.) supplemented with 10% heat-inactivated horse serum, 5% heat-inactivated fetal calf serum (Hyclone, Logan, Utah), 4 mM L-glutamine, streptomycin, and penicillin G (Fisher, Pittsburgh, Pa.). Cells were maintained at 37° C., in 5% CO₂ humid atmosphere.

The effects of various neuroprotective agents were examined in the PC12-αSyn cell line. To determine MPP⁺ neurotoxicity in α-synuclein- and naïve PC12 cells, cell death was measured by LDH release assays. The PC12-αSyn cells showed a significantly higher LDH release in response to an intermediate concentration (2.5 mM) of MPP⁺ than naive PC12 cells, but there was no significant difference between the two groups when treated with higher concentrations of MPP⁺. This indicated that below a threshold of “toxic overload”, α-synuclein overexpression provided a more susceptible environment to MPP⁺ insult. When the PC12-αSyn cells were pretreated with neuroprotective agents such as BAF, a pan-caspase inhibitor, this MPP⁺ susceptibility was significantly decreased demonstrating that the PC12-αSyn cell model is useful for studying neuroprotection in vitro.

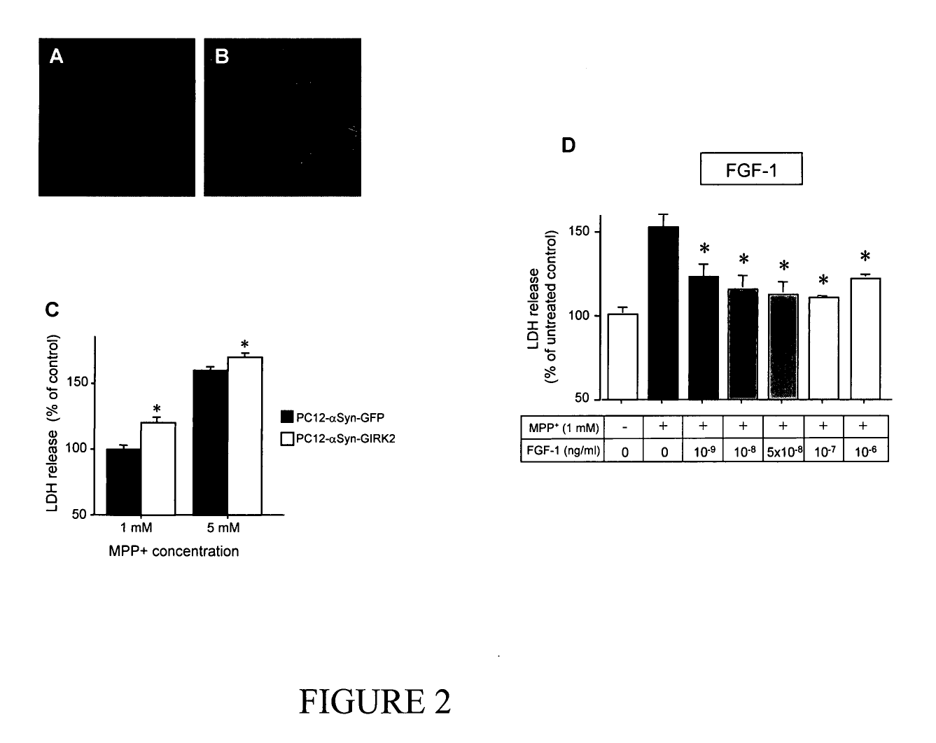

GIRK2 is known to be more expressed in A9 DA neurons and linked to degeneration of A9 DA neurons in the weaver mouse. We tested whether an increase in GIRK2 expression levels can modulate vulnerability to MPP⁺ in PC12-αSyn (FIGS. 2A and 2B). This cell culture model combines two features of PD-related pathogenetic mechanisms: MPP⁺ toxicity and α-synuclein overexpression. Overexpression of α-synuclein increased susceptibility of PC12 cells (PC12-αSyn) to MPP⁺ compared to control PC-12 cells. Lentivirus-mediated overexpression of GIRK2 in PC12-αSyn resulted in significantly increased vulnerability at low (1 mM) and high (5 mM) MPP⁺ concentrations compared to control (eGFP-transduced) cells (FIG. 2C). Thus, GIRK2 is a gene that is naturally overexpressed in the A9 DA neurons and contributes to increased susceptibility to MPP⁺ in the presence of α-synuclein.

To further investigate whether differentially expression proteins contribute to the survival differences between DA neuronal subtypes, other candidates from the comparative genetic profiles (Table 2) were tested. The results in Table 2 demonstrate that growth factor activity, especially FGF-related activity, is prominent in A9 dopaminergic neurons. FGF1 was chosen for further investigation because its mRNA expression level and that of its receptor, FGFR3, are relatively high (Table 1). Additionally, the FGF1 gene is contained in Parkinson's disease linkage region (Table 3). The data in FIG. 2D demonstrate that FGF1 was able to protect PC12-αSyn cells from MPP⁺ in a dose-dependent manner.

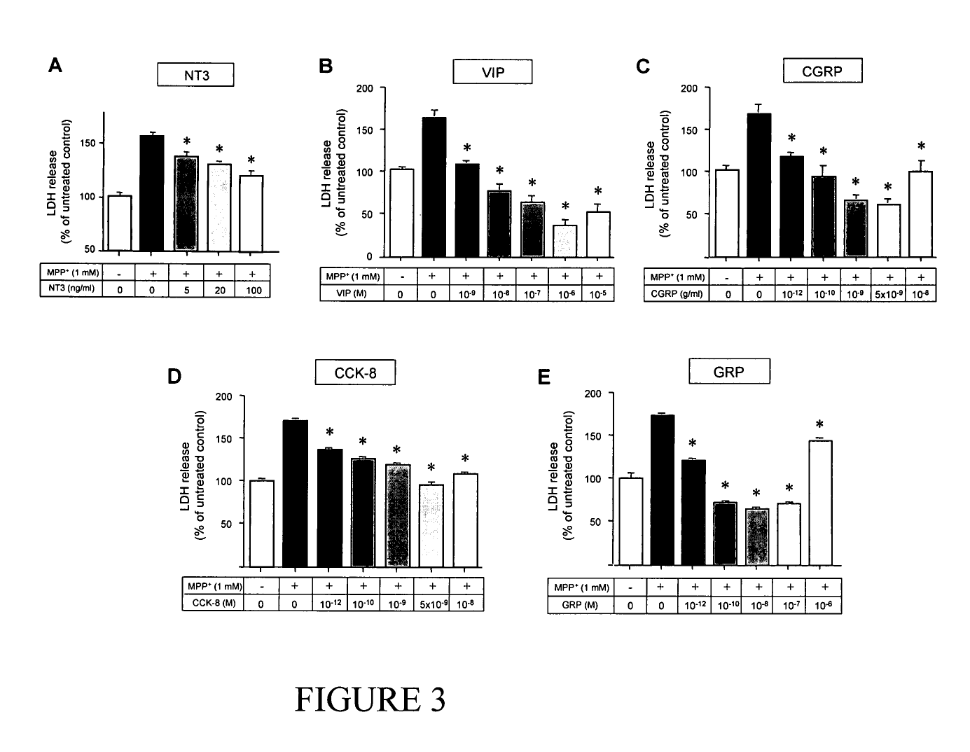

A10 DA neurons are less vulnerable in PD. Certain genes more highly expressed in A10 compared to A9 DA neurons may play a neuroprotective role. The neurotrophic factor, NT-3, which is known to have elevated expression in A10 dopaminergic neurons, was tested in the PC12-αSyn assay described above. NT-3 was able to protect PC12-α-Syn cells from MPP⁺ neurotoxicity in a dose-dependent manner (FIG. 3A).

The microarray analysis reveals that a group of neuropeptides is elevated in A10 dopaminergic neurons (Tables 1 and 2). Because neuropeptides are known to exert trophic effects, several were chosen to test their potential protective effects from MPP⁺ toxicity in PC12-αSyn cells. Vasoactive intestinal peptide (VIP), calcitonin/calcitonin gene-related peptide alpha (CGRP), cholecysokinin-8 (CCK-8), and gastrin releasing peptide (GRP) were individually applied to the test cells in the presence of 1 mM MPP⁺. All of these neuropeptides exhibited dose-dependent neuroprotective effects as determined by LDH assay (p<0.01; FIGS. 3B-3E).

Primary Ventral Mesencephalic Dopaminergic Neuronal (VMDA) Cell Culture

Primary cultured VMDA cells contain a mixed cell population of A9-like and A10-like DA neurons and can be used in the screening methods of the invention. These cultures have several advantages including providing authentic DA neurons cultured with their naturally occurring neighboring cells.

VMDA cultures were obtained from E15 Sprague-Dawley rat (Charles River, Mass.) ventral mesencephalon (VM). Tissue was mechanically dissociated with a pasteur pipette. The cells were resuspended in DMEM containing heat-inhibited horse serum (10%), glucose (6.0 mg/ml), penicillin, streptomycin, and 2 mM glutamine (Gibco). Cell suspensions containing 4×10⁵ cells were plated on a coverslip in a 24-well plate, precoated with a 1:500 diluted solution of polyornithine and fibronectin in 50 mM sodium borate (pH 7.4) overnight.

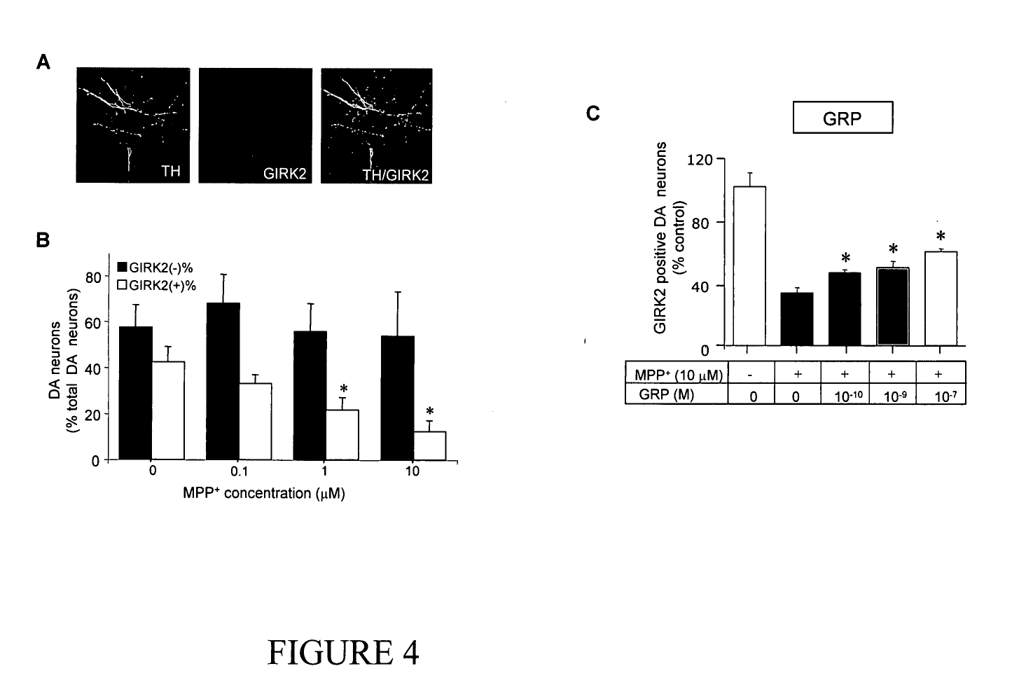

Primary ventral mesencephalic (VM) cultures were used to further delineate the protective effects of neuropeptides on dopaminergic neurons. These cultures contained a mixed cell population having approximately 40% of TH-positive neurons at 5 days in vitro (DIV) with both A9- and A10-like dopaminergic neurons (FIG. 4A). GIRK2-positive cells were more vulnerable to MPP⁺ compared to GIRK2-negative cells (FIG. 4B). The cultures were treated with MPP⁺ at day 5 for 48 hours using concentrations ranging from 0.1 to 10 μM. Cells were fixed with paraformaldehyde for immunostaining of TH and GIRK2 48 hours after MPP⁺ treatment. These results further demonstrate that increased vulnerability of neurons that express high levels of GIRK2.

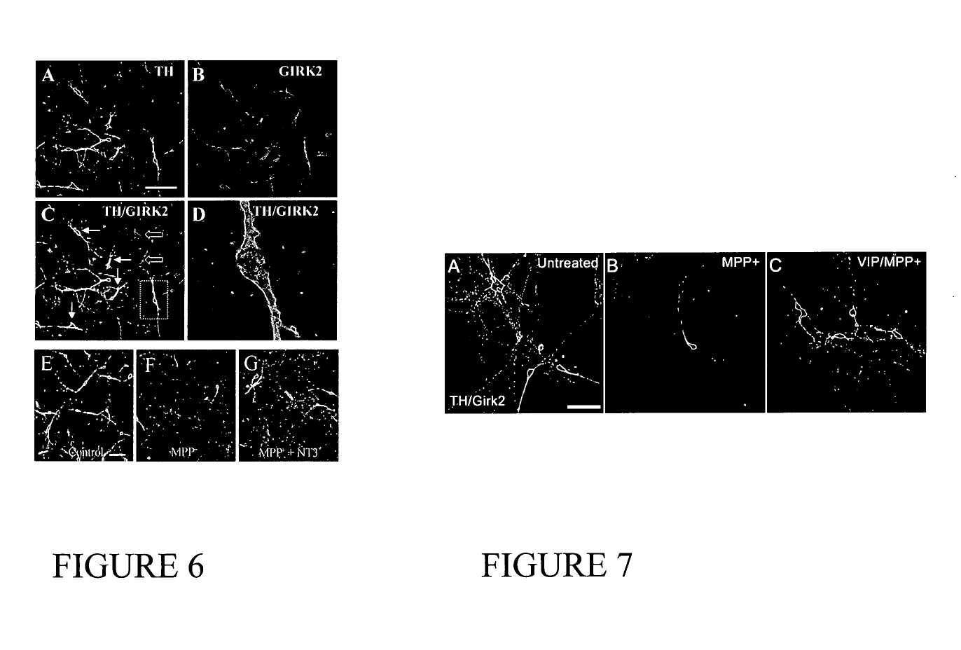

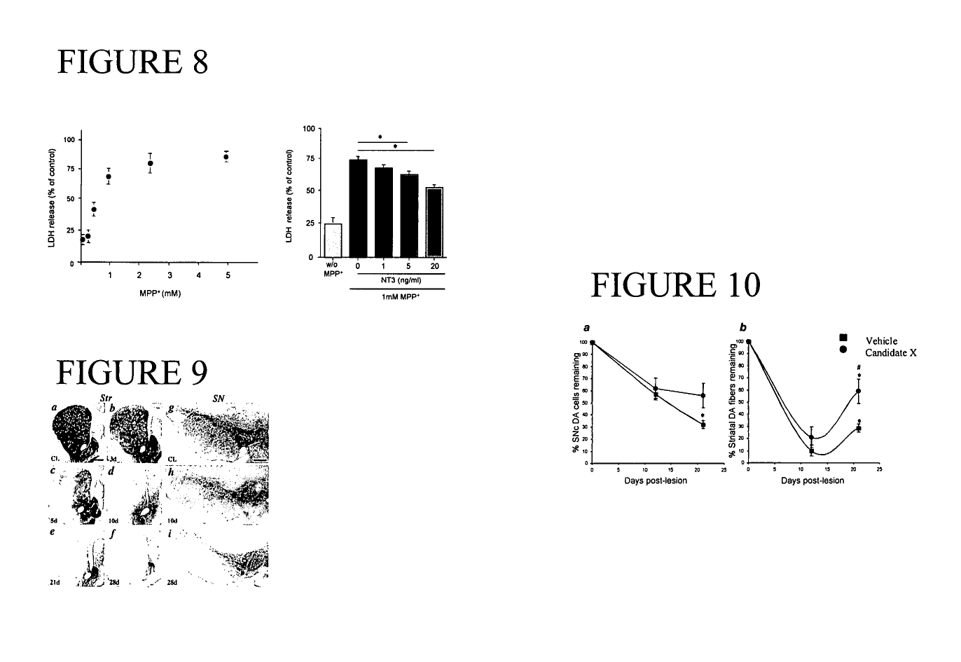

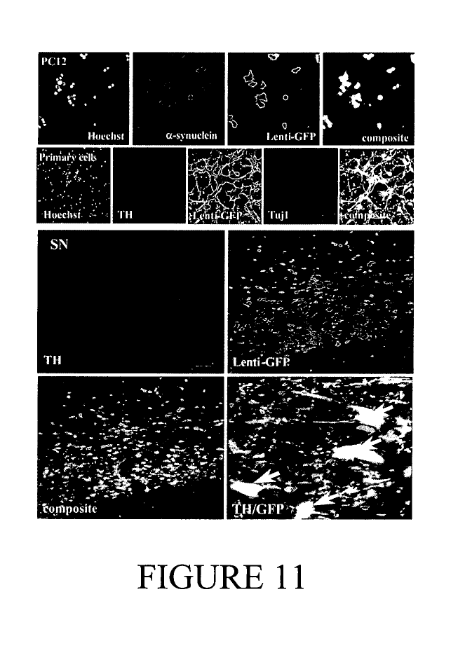

The neuroprotective effect of GRP, NT-3 and VIP was examined in these primary VM cell cultures. Each of the polypeptide factors, GRP (FIG. 4C), NT-3 (FIGS. 6A-6G), and VIP (FIGS. 8A-8C) exhibited a significant protective effect against MPP⁺ toxicity GIRK2-positive/TH-positive neurons. The cultures were pretreated with the polypeptide factors 2 hours prior to 10 μM MPP⁺ treatment.

We have determined the conditions of MPP⁺ toxicity in the VM cultures and demonstrate neuroprotective effects of neuropeptides typically more abundantly expressed by A10 DA neurons, when added to the culture medium (FIGS. 4, 6A-6G, and 7A-7C). These data show that VM cultures are a useful tool for testing neuroprotection as described herein.

Human Dopaminergic Neuron Precursor (DAN) Cells

DAN cells, which have been used to study neurodegeneration, can be used in the methods of this invention; they express multiple genes of Tables 4 and/or 5, and the expression of these genes following contact with a candidate compound can be measured. DAN cells exhibit the neurochemical characteristics of mesencephalic DA neurons, e.g. greater than 70% of DAN cells have robust immunoreactivity for dopamine, TH, DAT and vesicular monoamine transporter (VMAT) and more than 90% of DAN cells are neurons. DAN cells are sensitive to the neurotoxin MPP⁺. NT3, one of our candidate molecules from our microarray data, was shown to have a protective effect in MPP⁺ treated DAN cells, decreasing cell death as detected by lactate dehydrogenase (LDH) release assays (FIG. 8). These data suggest that our DAN cell system is appropriate to study the europrotection/vulnerability of our candidate molecules.

In Vivo Models of Parkinson's Disease

Several in vivo models of Parkinson's disease exist that mimic some of the clinical and pathological features of Parkinson's disease including, for example, L-dopa-responsive movement disorder, a chronically progressive loss of dopaminergic neurons, and the presence of Lewy body (LB)-like inclusions. These models can be used to confirm results obtained using the methods of the invention. In these models, the neurotoxins 6-OHDA and MPTP are commonly used to induce dopaminergic neuronal cell death leading to parkinsonism. In rats, intrastriatal 6-OHDA causes progressive degeneration of dopaminergic neurons in SN similar to the slope of the time-course seen in human PD. MPTP also causes selective dopaminergic neuronal loss in brain areas similar to idiopathic PD in mice and primates. In primates, intracarotid administered MPTP is used for a rapid destruction of dopaminergic neurons in the substantia nigra followed by symptoms of parkinsonism. Since this model lacks typical aspects of slowly progessive neurodegeneration, it is limited in modeling the pathophysiology of Parkinson's disease in patients. We have further improved this model by repeated systemic administration of low doses of MPTP to induce a parkinsonian syndrome that shares many characteristics with idiopathic Parkinson's disease. This model is useful for testing the effects of neuroprotective molecules in primates.

Another model for Parkinson's disease is based on the injection of α-synuclein-expressing recombinant Adeno Associated Viruses (rAAV-α-syn) into the striatum of rodents and primates. Since α-synuclein is a major component of LBs, overexpression of wild type or mutant (A53T and A30P) α-synuclein selectively induces an α-synucleinopathy including LB formation in targeted DA neurons.

Progressive 6-OHDA Lesioned Rats

Female Sprague Dawley rats (200-250 g, Charles River, Wilmington, Mass.) receive unilateral intrastriatal stereotaxic injections of 6-OHDA (Sigma, St. Louis, USA) using a 10 μl Hamilton syringe. Intramuscular injections of Acepromazine (3.3 mg/kg, PromAce, Fort Dodge, Iowa) and atropine sulfate (0.2 mg/kg, Phoenix Pharmaceuticals, St. Joseph, Mo.) is given 10 minutes before animals will be anesthetized with ketamine/xylazine (60 mg/kg and 3 mg/kg respectively, i.m.). A concentration of 3.0 μg/μl free base 6-OHDA dissolved in 0.2% ascorbic acid/saline (Sigma) is injected into 3 locations (2.5 μl/site, total dose 22.5 μg) in the right striatum at the following coordinates (calculated from bregma): site 1, AP+1.3, L−2.8, DV−4.5, IB−2.3; site 2, AP+0.2, L−3.0, DV−5.0, IB−2.3; site 3, AP−0.6, L−4.0, DV−5.5, IB−2.3 mm. FIGS. 9A-9F shows the progressive time course of dopaminergic fiber loss in the striatum of 6-OHDA-lesioned rats. The loss of striatal dopaminergic fibers is accompanied by a progressive degeneration of dopaminergic neuronal cell bodies in the substantia niga (FIGS. 9G-I). These results are quantified in FIGS. 10A-10B.

Behavioral Test for Rat Models

Behavioral bioassays of the DA system may be used as follows. For the head bias assessment, the animals are placed into observation cages in a quiet room with dim lighting and allowed to habituate for 5 minutes. For one minute, the animals are scored according to exhibited head bias to one side or the other during each second if the animals' heads may be 10° or more out of line with their shoulders in either direction. The seconds are monitored with a digital metronome. Each animal will be scored three times, all of which occurred after a single habituation period. The scores are then summed between periods, and the percentage of bias to the contralateral side is calculated and reported as the final score.

For the cylinder test, the animals is placed inside an 18-gallon cylinder (Nalgene, Rochester, N.Y.) between two mirrors set up at right angles to each other to facilitate scoring of movements made on sides of the cylinder not facing the observer. The rats are videotaped for the 3 minutes immediately following placement. After the test, an observer scores each instance in which a rat beginning with all four paws on the floor places one or both forelimbs on the wall of the cylinder. The score is recorded as the fraction of total contacts in which the paw contralateral to the lesion touched the wall first.

For the apomorphine assessment, the animals receive 0.1 mg/kg subcutaneous injection of apomorphine. The animals are placed in the same recording system as in the amphetamine test, and are monitored for 40 minutes. Follow-up assessments are conducted with the same protocol.

Lesioned animals are selected for study by examination of rotational behavior in response to a 4 mg/kg intraperitoneal (i.p.) injection of amphetamime. The animals are randomized and placed in automated rotometer bowls and left and right full-body turns are monitored via a computerized activity monitor system for 90 minutes. Follow-up amphetamine rotation studies may be conducted following the same protocol.

Identification of Candidate Compounds Useful for Treating Parkinson's Disease

A candidate compound that is beneficial for treating or preventing PD can be identified using the methods of this invention. A candidate compound can be identified for its ability to reduce the expression or biological activity of a gene listed in Table 4 or increase the expression or biological activity of a gene listed in Table 5, or both. Candidate compounds that modulate the expression level or biological activity of the polypeptide of the invention by at least 10%, 20%, 30%, 40%, 50%, 60%, 70%, 80%, 90%, 95%, 100%, or more relative to an untreated control not contacted with the candidate compound are identified as compounds useful for treating and preventing PD.

Customized High Throughput Expression Screening Systems

Customized “gene chips” provide a high-throughput system for screening large numbers of samples obtained from in vitro or in vivo tissue sources. Genes involved in the pathology of PD are first selected (including those listed in Tables 4 and 5) and a customized gene chip is next created by immobilizing the appropriate nucleic acid probes on a solid support. Selected nucleic acid probes are immobilized onto predetermined regions of a solid support by any suitable techniques that affect a stable association (i.e., the nucleic acids remain localized to the predetermined region under hybridization and washing conditions) of the probes with the surface of a solid support.

The nucleic acids may be covalently associated with or non-covalently attached to the support surface. Examples of non-covalent association include binding as a result of non-specific adsorption, ionic, hydrophobic, or hydrogen bonding interactions. Covalent association involves formation of chemical bond between the nucleic acids and a functional group present on the surface of a support. The functional group may be naturally occurring or introduced as a linker. Non-limiting functional groups include but are not limited to hydroxyl, amine, thiol and amide. Exemplary techniques applicable for covalent immobilization of polynucleotide probes include, but are not limited to, UV cross-linking or other light-directed chemical coupling, and mechanically directed coupling (see, for example, U.S. Pat. Nos. 5,837,832, 5,143,854, 5,800,992, WO 92/10092, WO 93/09668, and WO 97/10365). A preferred method is to link one of the termini of a nucleic acid probe to the support surface via a single covalent bond. Such configuration permits high hybridization efficiencies as the probes have a greater degree of freedom and are available for complex interactions with complementary targets.

A probe may be associated with the surface directly or may be indirectly associated by an intermediate “spacer” moiety. Such a spacer can be of any material, e.g., any of a variety of materials which are conventional in the art. In one embodiment, the spacer is an organic moiety having, e.g., about 5-20 Cs. In another embodiment, the spacer is a nucleic acid (of any of the types describes elsewhere herein) which does not undergo specific interaction or association with, e.g., a target nucleic acid of the invention.

Typically, each array is generated by depositing a plurality of probe samples either manually or more commonly using an automated device, which spots samples onto a number of predefined regions in a serial operation. A variety of automated spotting devices are commonly employed for production of polynucleotide arrays. Such devices include piezo or ink-jet devices, automated micro-pipetters and any of those devices that are commercially available (e.g. Beckman Biomek 2000). The total number of probe samples spotted on the support will vary depending on the number of different polynucleotide probes one wish to display on the surface, as well as the number of control probes, which may be desirable depending on the particular application in which the subject array is to be employed. Generally, the array comprises at least about 20 distinct polynucleotides, usually at least about 100 polynucleotides, preferably about 1000 polynucleotides, more preferably about 10,000 polynucleotides, but will usually not exceed 100,000 polynucleotides, wherein each polynucleotide is complementary to a target nucleic acid. The polynucleotide spots may take a variety of configurations ranging from simple to complex, depending on the intended use of the array. The probes may be spotted in any convenient pattern across or over the surface of the array so as to from a grid, a circular, ellipsoid, oval or some other analogously curved shape. Within a predetermined region, the probes are deposited in an amount sufficient to provided adequate hybridization and detection of target nucleic acids during a hybridization assay.

Preferably, a predetermined region comprises at least 2, preferably at least 100 single-stranded polynucleotides, more preferably about 1000 single-stranded polynucleotides, and will usually not exceed 10,000 polynucleotide probes, that are complementary to a nucleic acid of the invention. Typically, a predetermined region is spotted with at least 2, usually at least 100 single-stranded polynucleotides of identical sequences. The predetermined region generally has an average size ranging from about 0.01 cm² to about 1 cm².

The substrates of the subject arrays may be manufactured from a variety of materials. In general, the materials with which the support is fabricated exhibit a low level of non-specific binding during hybridization assay. A preferred solid support is made from one or more of the following types of materials: nitrocellulose, nylon, polypropylene, glass, and silicon. The materials may be flexible or rigid. A flexible substrate is capable of being bent, folded, twisted or similarly manipulated, without breaking. A rigid substrate is one that is stiff or inflexible and prone to breakage. As such, the rigid substrates of the subject arrays are sufficient to provide physical support and structure to the polymeric targets present thereon under the assay conditions in which the arrays are employed, particularly under high throughput assay conditions. Exemplary materials suitable for fabricating flexible support include a diversity of membranous materials, such as nitrocellulose, nylon or derivatives thereof, and plastics (e.g. polytetrafluoroethylene, polypropylene, polystyrene, polycarbonate, and blends thereof). Examples of materials suitable for making rigid support include but are not limited to glass, semi-conductors such as silicon and germanium, metals such as platinum and gold. The solid support on which arrays of polynucleotide probes are attached comprises at least one surface, which may be smooth or substantially planar, or with irregularities such as depressions or elevations.

The surface on which the pattern of probes is deposited may be modified with one or more different layers of compounds that serve to modify the properties of the surface in a desirable manner. Modification layers coated on the solid support may comprise inorganic layers made of, e.g. metals, metal oxides, or organic layers composed of polymers or small organic molecules and the like. Polymeric layers of interest include layers of peptides, proteins, polysaccharides, lipids, phospholipids, polyurethanes, polyesters, polycarbonates, polyureas, polyamides, polyethyleneamines, polyarylene sulfates, polysiloxanes, polyimides, polyacetates and the like, where the polymers may be hetero- or homopolymeric, and may or may not be conjugated to functional moieties. Arrays of polynucleotide probes provide an effective means of detecting or monitoring expression of a multiplicity of genes and may be used in a wide variety of circumstances including identifying compounds useful for treating or preventing a neurodegenerative disorder, identification and quantification of differential gene expression between at least two samples, such as, for example a control sample and a sample contacted with a polypeptide of the invention, and/or screening for compositions of the invention that upregulate or downregulate the expression or alter the pattern of expression of particular genes that are involved in neurodegenerative disorders.

Where desired, the resulting transcribed nucleic acids may be amplified prior to hybridization. One of skill in the art will appreciate that whichever amplification method is used, if a quantitative result is desired, caution must be taken to use a method that maintains or controls for the relative copies of the amplified nucleic acids. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The subject array may also include probes specific to the internal standard for quantification of the amplified nucleic acid. Detailed protocols for quantitative PCR are provided in PCR Protocols, A Guide to Methods and Applications, Innis et al., Academic Press, Inc. N.Y., 1990.

Suitable hybridization conditions for the practice of the present invention are such that the recognition interaction between the probe and target is both sufficiently specific and sufficiently stable. Hybridization reactions can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, for example, Sambrook, et al., supra.

For a convenient detection of the probe-target complexes formed during the hybridization assay, the target nucleic acids can be conjugated to a detectable label. Detectable labels suitable for use in the present invention include any composition detectable by spectroscopic, photochemical, biochemical, immunochemical, electrical, optical or chemical means. A wide variety of appropriate detectable labels are known in the art, which include luminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase, peroxidase, or avidin/biotin complex.

The labels may be incorporated by any of a number of means well known to those of skill in the art. In one aspect, the label is simultaneously incorporated during the amplification step in the preparation of the target nucleic acids. Thus, for example, polymerase chain reaction (PCR) with labeled primers or labeled nucleotides can provide a labeled amplification product In a separate aspect, transcription reaction, as described above, using a labeled nucleotide (e.g. fluorescein-labeled UTP and/or CTP) or a labeled primer, incorporates a detectable label into the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

The detection methods used to determine where hybridization has taken place and/or to quantify the hybridization intensity will typically depend upon the label selected above. For example, radiolabels may be detected using photographic film or phosphoimager (for detecting and quantifying ³²P incorporation). Fluorescent markers may be detected and quantified using a photodetector to detect emitted light (see U.S. Pat. No. 5,143,854 for an exemplary apparatus). Enzymatic labels are typically detected by providing the enzyme with a substrate and measuring the reaction product produced by the action of the enzyme on the substrate; and finally colorimetric labels are detected by simply visualizing the colored label.

The detection method provides a positional localization of the region where hybridization has taken place. The position of the hybridized region correlates to the specific sequence of the probe, and hence the identify of the gene transcript expressed in the test subject. The detection methods also yield quantitative measurement of the level of hybridization intensity at each hybridized region, and thus a direct measurement of the level of expression of a given gene transcript. A collection of the data indicating the regions of hybridization present on an array and their respective intensities constitutes a “hybridization pattern” that is representative of a multiplicity of expressed gene transcripts of a subject. Any discrepancies detected in the hybridization patterns generated by hybridizing target nucleic acids derived from different subjects are indicative of differential expression of a multiplicity of gene transcripts of these subjects.

One of skill in the art, however, will appreciate that hybridization signals will vary in strength with efficiency of hybridization, the amount of label on the target nucleic acid and the amount of particular target nucleic acid in the sample. Typically target nucleic acids present at very low levels (e.g., <1 pmol) will show a weak signal. In evaluating the hybridization data, a threshold intensity value may be selected below which a signal is not counted as being essentially indistinguishable from background. In addition, the provision of appropriate controls permits a more detailed analysis that controls for variations in hybridization conditions, cell health, non-specific binding and the like. The subjects employed for the comparative hybridization analysis may be cells treated with or without external or internal stimuli. Thus, the comparative hybridization analysis using the arrays described herein can be employed to monitor gene expression in a wide variety of contexts. Such analysis may be extended to detecting differential expression of genes between diseased and normal tissues, or amongst cells that are subjected to various environmental stimuli or candidate drugs, such as the polypeptides of the present invention.

Assays for Measuring Expression Levels

The screening methods of the invention may be used to identify candidate compounds that modulate the expression levels of any of the polypeptides listed in Table 4 or Table 5 by at least 10%, 20%, 30%, 40%, 50%, 60%, 70%, 80%, 90%, 95%, or 100% relative to an untreated control. According to one approach, candidate compounds are added at varying concentrations to the culture medium of cells expressing the polypeptide of Table 4 or Table 5. Gene expression of the polypeptide is then measured, for example, by standard Northern blot analysis (Ausubel et al., supra), using any appropriate fragment prepared from the nucleic acid molecule encoding the polypeptide as a hybridization probe or by real time PCR with appropriate primers. The level of gene expression in the presence of the candidate compound is compared to the level measured in a control culture medium lacking the candidate molecule. If desired, the effect of candidate compounds may, in the alternative, be measured at the protein level using the same general approach and standard immunological techniques, such as Western blotting or immunoprecipitation with an antibody specific to the polypeptide for example. For example, immunoassays may be used to detect or monitor the level of the polypeptide from Table 4 or Table 5. Polyclonal or monoclonal antibodies which are capable of binding to such polypeptides may be used in any standard immunoassay format (e.g., ELISA or RIA assay) to measure protein levels of the polypeptide. The polypeptide of the invention can also be measured using mass spectroscopy, high performance liquid chromatography, spectrophotometric or fluorometric techniques, or combinations thereof.

Reporter Gene Assays

Expression of a reporter gene that is operably linked to the promoter of a gene identified in Tables 4 or 5, (e.g., an NT-3, VIP, CGRP, CCK-8, or GRP promoter) can also be used to identify a candidate compound for treating or preventing PD. Assays employing the detection of reporter gene products are extremely sensitive and readily amenable to automation, hence making them ideal for the design of high-throughput screens. Assays for reporter genes may employ, for example, calorimetric, chemiluminescent, or fluorometric detection of reporter gene products. Many varieties of plasmid and viral vectors containing reporter gene cassettes are easily obtained. Such vectors contain cassettes encoding reporter genes such as lacZ/β-galactosidase, green fluorescent protein, and luciferase, among others. A genomic DNA fragment carrying a selected transcriptional control region (e.g., a promoter and/or enhancer) is first cloned using standard approaches (such as those described by Ausubel et al. (supra). The DNA carrying the selected transcriptional control region is then inserted, by DNA subcloning, into a reporter vector, thereby placing a vector-encoded reporter gene under the control of that transcriptional control region. The activity of the selected transcriptional control region operably linked to the reporter gene can then be directly observed and quantified as a function of reporter gene activity in a reporter gene assay.

In one embodiment, for example, the transcriptional control region could be cloned upstream from a luciferase reporter gene within a reporter vector. This could be introduced into the test cells, along with an internal control reporter vector (e.g., a lacZ gene under the transcriptional regulation of the β-actin promoter). After the cells are exposed to the test compounds, reporter gene activity is measured and the reporter gene activity is normalized to internal control reporter gene activity.

Assays Measuring Biological Activity

Based on this invention, a candidate compound may be tested for its ability to modulate the biological activity of one or more polypeptides listed in Table 4 or Table 5 in cells that naturally express such a polypeptide, after transfection with a cDNA for this polypeptide, or in cell-free solutions containing the polypeptide, as described further below. Accordingly, candidate compounds are first contacted with a polypeptide from either table, having some level of a characteristic biological activity (including cell survival). The exact level of activity is unimportant and may be at least 10%, 20%, 30%, 40%, 50%, 60%, 70%, 80%, 90%, 100%, or more than 100% of the biological activity of the naturally-occurring, wild-type polypeptide. The effect of a candidate compound on the activity of the polypeptide can be tested by radioactive and non-radiaoactive binding assays, competition assays, and receptor signaling assays.

In one example, a cell (e.g., a primary VMDA) expressing the polypeptide of Table 4 or Table 5 is contacted with a candidate compound, after which the biological activity (e.g., survival of a cell or any one of the activities associated with the naturally-occurring polypeptide) of the polypeptide is measured in the cell. In another example, contacting between candidate compounds and polypeptides occurs in a cell-free system or in an animal, and biological activity is then determined. Biological activity may be determined using any standard method, including those described herein. A candidate compound that modulates such biological activity relative to that of the same polypeptide in a cell not contacted with the candidate compound, identifies the candidate compound as being useful for treating or preventing neurodegenerative disorders.

Candidate compounds of the present invention may also be identified based on their ability to increase the growth or survival of cells (e.g., dopaminergic cells) that express one of a panel of target polypeptides. For example, a candidate compound may be contacted with a plurality of cell populations, such that each contacting event is segregated from the others. Each population of cells expresses a polypeptide listed in Table 4 or Table 5. A candidate compound that increases the growth or survival of at least two populations of cells expressing such polypeptides, relative to the growth or survival of control populations not contacted with the candidate compound, is identified as a compound having the ability to treat or prevent neurodegenerative disorders. Optionally, assays measuring cell growth or cell survival may also be employed to confirm that a candidate compound identified by any of the other assays of the invention (e.g., assays detecting expression levels or biological activity (other than cell survival) of the polypeptides) can effectively increase cell survival.

If the contacting event occurs in vivo, the biological activity of the candidate compound may be assessed by determining the survival of treated animals relative to untreated animals, the reduction in neurodegenerative symptoms (e.g., neuronal atrophy) in treated animals relative to untreated animals, or both.

Candidate Compounds

Candidate compounds (e.g., organic molecules, peptides, peptide mimetics, polypeptides, nucleic acids, or antibodies) may be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (see e.g., Lam, Anticancer Drug Des. 12:145, 1997).

Examples of methods for the synthesis of molecular libraries can be found in the art, for example in: DeWitt et al., Proc. Natl. Acad Sci. USA. 90:6909, 1993; Erb et al., Proc. Natl. Acad Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Libraries of compounds may be presented in solution (Houghten, Biotechniques 13:412-421, 1992), or on beads (Lam, Nature 354:82-84, 1991), chips (Fodor, Nature 364:555-556, 1993), bacteria (Ladner U.S. Pat. No. 5,223,409), spores (Ladner U.S. Pat. No. 5,223,409), plasmids (Cull et al., Proc. Natl. Acad. Sci. USA 89:1865-1869, 1992) or on phage (Scott et al., Science 249:386-390, 1990; Devlin, Science 249:404-406, 1990; Cwirla et al., Proc. Natl. Acad. Sci. 87:6378-6382 1990; Felici, J. Mol. Biol. 222:301-310, 1991).

Optionally, either the polypeptide of Table 4 or Table 5 or the candidate compound may include a label or tag that facilitates their isolation. For polypeptides, an exemplary tag of this type is a poly-histidine sequence generally containing around six histidine residues that permits the isolation of a compound so labeled by means of nickel chelation. Other labels and tags, such as the FLAG tag (Eastman Kodak, Rochester, N.Y.), are well known and are routinely used in the art. Small molecules may be radiolabeled for detection.

Candidate Compounds: Polypeptides and Proteins

For their use in the present invention as candidate compounds, recombinant polypeptides, particularly polypeptides of Table YY, may be produced using any standard technique known in the art. Following their production, these polypeptides are useful, for example, for the identification of therapeutic compounds using the methods described herein.

Host cells, such as yeast, bacterial, mammalian, and insect cells, may produce any of the polynucleotides of the present invention. These cells may produce such polynucleotides endogenously or may alternatively be genetically engineered to do so. Polynucleotides may be introduced into host cells using any standard method known in the art, including, for example, calcium phosphate transfection, DEAE-dextran mediated transfection, transvection, microinjection, cationic lipid-mediated transfection, electroporation, transduction, ballistic introduction, and infection or fusion with carriers such as liposomes, micelles, ghost cells, and protoplasts.

In general, any expression system or vector that is able to maintain, propagate, or express a polynucleotide to produce a polypeptide in a host may be used. These include chromosomal, episomal, and virus-derived systems such as vector-derived bacterial plasmids, bacteriophages, transposons, yeast episomes, insertion elements, yeast chromosomal elements, viruses (such as baculoviruses, papova viruses (e.g., SV40), vaccinia viruses, adenoviruses, fowl pox viruses, pseudorabies viruses, and retroviruses), and vectors derived from combinations thereof, such as those derived from plasmid and bacteriophage genetic elements, such as cosmids and phagemids. Preferred expression vectors include, but are not limited to, pcDNA3 (Invitrogen) and pSVL (Pharmacia Biotech). Other exemplary expression vectors include pSPORT vectors, pGEM vectors (Promega), pPROEXvectors (LTI, Bethesda, Md.), Bluescript vectors (Stratagene), pQE vectors (Qiagen), pSE420 (Invitrogen), and pYES2 (Invitrogen). Optionally, the expression systems may contain control regions that facilitate or regulate expression. The appropriate polynucleotide may be inserted into an expression system by any of a variety of well-known and routine techniques, including transformation, transfection, electroporation, nuclear injection, or fusion with carriers such as liposomes, micelles, ghost cells, and protoplasts.

Expression systems of the invention include bacterial, yeast, fungal, plant, insect, invertebrate, vertebrate, and mammalian cells systems. If a eukaryotic expression vector is employed, then the appropriate host cell is any eukaryotic cell capable of expressing the cloned sequence. Preferably, eukaryotic cells are cells of higher eukaryotes. Suitable eukaryotic cells include non-human mammalian tissue culture cells and human tissue culture cells. Preferred host cells include insect cells, HeLa cells, Chinese hamster ovary cells (CHO cells), African green monkey kidney cells (COS cells), human 293 cells, murine embryonal stem (ES) cells, and murine 3T3 fibroblasts. The propagation of such cells in cell culture is standard in the art. Yeast hosts may also be employed as a host cell. Preferred yeast cells include the genera Saccharomyces, Pichia, and Kluveromyces. Preferred yeast hosts are Saccharomyces cerevisiae and Pichia pastoris. Yeast vectors may contain any of the following elements: an origin of replication sequence from a 2T yeast plasmid, an autonomous replication sequence (ARS), a promoter region, sequences for polyadenylation, sequences for transcription termination, and a selectable marker gene. Shuttle vectors for replication in both yeast and E. coli are also included herein.

Alternatively, insect cells may be used as host cells. In a preferred embodiment, the polypeptides of the invention are expressed using a baculovirus expression system. The Bac-to-Bac complete baculovirus expression system (Invitrogen) may be used, for example, for protein production in insect cells.

Expression of polypeptides in prokaryotes is most often carried out in E. coli with vectors containing constitutive or inducible promoters directing the expression of either fusion or non-fusion proteins. Fusion vectors add a number of amino acids to a polypeptide encoded therein, usually to the amino terminus of the recombinant polypeptide. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant polypeptide to enable separation of the recombinant protein from the fusion moiety subsequent to purification of the fusion protein. Such enzymes, and their cognate recognition sequences, include Factor Xa, thrombin, and enterokinase. Typical fusion expression vectors include pGEX, pMAL, and pRIT5, which fuse glutathione S-transferase (GST), maltose E binding protein, and protein A, respectively, to the target recombinant protein.

The polypeptides of the present invention may also be expressed at the surface of cells, which are then harvested prior to use in the screening assay. If the polypeptide is secreted into the medium, the medium may be recovered in order to recover and purify the polypeptide. If produced intracellularly, the cells must first be lysed before the polypeptide is recovered. Polypeptides of the present invention may be recovered and purified from recombinant cell cultures or lysates by well-known methods including ammonium sulfate or ethanol precipitation, acid extraction, anion or cation exchange chromatography, phosphocellulose chromatography, hydrophobic interaction chromatography, affinity chromatography, hydroxyapatite chromatography, and lectin chromatography. Most preferably, high performance liquid chromatography is employed for purification. Well-known techniques for refolding proteins may be employed to regenerate active conformation when the polypeptide is denatured during intracellular synthesis, isolation, and/or purification.

Optionally, the polypeptides of the present invention may be prepared by chemical synthesis using, for example, automated peptide synthesizers.

Materials and Methods

Laser Capture Microdissection (LCM)

Tissue Preparation

Adult C57/B6 mice (Jackson Laboratory, West Grove, Pa.) were anesthetized with intraperitoneal (i.p.) sodium pentobarbital (300 mg/kg) and decapitated. The brain was removed, snap-frozen in dry ice-cooled 2-methylbutane (−60° C.), and embedded in frozen tissue medium (Tissue Tek OTC, Sakura Finetek USA, Torrance, Calif.). Twelve micron-thick coronal sections of the midbrain were cut using a cryostat, mounted on LCM slides (Arcturus), and immediately stored at −70° C.

Quick Immunostaining and Dehydration of Sections

A quick immunostaining protocol against tyrosine hydroxylase (TH) was used to identify the dopaminergic neurons to be captured. The tissue sections will be fixed in cold 70% ethanol for 15 seconds, followed by 5 minutes fixation in cold acetone. Slides were washed in PBS, incubated with rabbit anti-TH (Pel-Freez Biologicals, Rogers, Ark.; 1:25) for 5 min, washed in PBS, and exposed to biotinylated anti-rabbit antibody (Vector Laboratories, Burlingame, Calif.; 1:300) for 5 minutes. Slides were washed in PBS, incubated in ABC-horseradish peroxidase enzyme complex (Vectastain, Vector Laboratories) for 5 min and the staining was detected with the substrate, diaminobenzidine (DAB). Sections were subsequently dehydrated in graded ethanol solution (1 min each in water, 70% ethanol, 95% ethanol, 100% ethanol, and twice for 5 min in xylene).

LCM of Mouse Midbrain Tissue

The PixCell II System (Arcturus Engineering, Inc, Mountain View, Calif.) was used for LCM. Approximately one thousand neurons were captured in each region of A9 and A10 in each animal. Since ventrolateral A9 cells are the most vulnerable and medial A10 cells are the most resistant to degeneration, only ventrolateral A9 (ventrolateral SNc, substantia nigra pars reticulata (SNr), substantia nigra pars lateralis (SNpl)) and medial A10 cells (central linear nucleus (CLi), interfascicular nucleus (IF), medial VTA, medial nucleus paranigralis (PN), medial nucleus parabrachialis pigmentosus (PBN)) were microdissected (FIGS. 1A-1D).

Affymetrix GeneChip Microarrays

Sample and Array Processing Total RNA was extracted from the individual samples using the PicoPure RNA isolation kit (Arcturus, Mountain View, Calif.). RNA quality was assessed by electrophoresis using the Agilent Bioanalyzer 2100 and spectrophotometric analysis prior to sample processing. Nanogram quantities of total RNA from each sample was used to generate a high fidelity cDNA, which is modified at the 3′ end to contain an initiation site for T7 RNA polymerase. Upon completion of cDNA synthesis all of the product was used in an in vitro transcription (IVT) reaction to generate aRNA. Up to 2 ug of aRNA was used for a second round of amplification that was initiated by random hexamer priming for first strand cDNA synthesis. The second round IVT contained biotinylated UTP and CTP which are utilized for detection following hybridization to the oligonucleotide microarray. 20 μg of full-length cRNA, from both control and enriched samples, were fragmented and hybridized to GeneChip arrays following the manufacturer's protocol. All samples were subjected to gene expression analysis via the Affymetrix Murine 430A high-density oligonucleotide array, which queries 22,000 mouse probe sets. Protocols for target hybridization, washing and staining were performed as per the manufacturer's protocol (http://www.affymetrix.com).

Data Normalization and Statistical Analysis

Several complementary data analysis approaches were used to identify differentially expressed genes. The Gene Chip Operating System 1.0 (GCOS, Affymetrix) was employed to generate one approach to comparative analysis presented in this study. Distinct algorithms were used to determine the absolute call, which distinguishes the presence or absence of a transcript, the differential change in gene expression and the magnitude of change, which is represented as signal log ratio (on a log base 2 scale). The mathematical definitions for each of these algorithms can be found in the GCOS data analysis guide. Two additional low level analysis methods were applied to all data sets outside of the Affymetrix normalization schema. Iobion's GeneTraffic MULTI was used to perform Robust Multi-Chip Analysis (RMA), which is a median polishing algorithm used in conjunction with both background subtraction and quantile normalization approaches. For each normalization approach, statistical analysis using the Significance Analysis Tool set in GeneTraffic was utilized. A two class unpaired analytical approach employing Benjamini-Hochberg correction for false discovery rate (FDR) was used for all probe level normalized data. Gene lists of differentially expressed genes were generated from this output for functional analysis. All data were organized in a central database in the University of Rochester Functional Genomics Center and is accessible through the following URL (www.fgc.urmc.rochester). Following each of these normalization approaches all genes differentially expressed were clustered based on biological relevance utilizing both hierarchical and K-means clustering techniques. Lastly, PathwayAssist (Iobion Informatics) was employed to build functional networks of differentially expressed genes.

Realtime PCR for Candidate Gene Validation

RNA samples from A9 and A10 DA neurons obtained from LCM were reverse-transcribed into cDNA using Sensiscript reverse transcriptase (Qiagen, Valencia, Calif.) and oligo dT as the primer. PCR reactions were set up in 25 μl reaction volume using SYBR Green PCR Master Mix (Applied Biosystems, Foster City, Calif.). Primers for each candidate gene were designed using MacVacter 7.0 and used with final concentration of 250 nM. For each primer pairs, duplicates of three to five independently collected A9 and A10 samples were compared to quantify relative gene expression differences between these cells using 2^{-ΔΔCT} method. Beta-actin was used as an internal control gene and primers for candidate genes with approximately equal (within 5% difference) amplification efficiency to that of the internal control were chosen.

Gene Transfer Using Lentivirus

Recombinant viruses, such as herpes simplex virus (HSV-1) and derivatives, Adenovirus (Ad), Adeno Associated Virus (AAV) and Lentivirus (LV) are effective vehicles for gene transfer to the adult CNS. Among these viruses, the LVs have several advantages, which make them an attractive tool for gene delivery to the brain. This includes a large cloning capacity (up to 9 kbp), stable integration into the genome of non-dividing cells, long-term gene expression, and high transduction efficiency of cells in both striatum and substantia nigra. The current third generation of LVs used in our studies has several features to reduce the risk of recombination events, such as the elimination of about 60% of the viral genome, the generation of virus particles with a four plasmid transfection system, and the introduction of self-inactivation. The efficiency of the LVs was also improved by pseudotyping with the vesicular stomatitis virus (VSV-G) envelope protein and the introduction of transcriptional and transduction enhancers into the virus backbone, such as the woodchuck virus regulatory element (WPRE) and a pol-derived poly pyrimidine tract (cPPT), respectively. Recombinant LVs have been successfully used in a variety of settings with high transduction and expression efficiencies including the brain. In particular, recombinant LVs expressing GDNF have been effective as a neuroprotective treatment in both rodent and primate models of PD.

Construction of Lentiviral Vectors

Mouse GIRK2 cDNA was cloned into lentiviral vector, pRRL.cPPT.PGK.GFP. W.Sin-18 vector. Virus constructs was confirmed by sequence analyses.

Production of Lentiviral Vectors and Cell Transduction

High titer of infectious lentiviral particles were produced in 293 T cells using a four-plasmid transfection protocol (Current Protocols in Neuroscience, 2000, 4.21.1-4.21.12). The packaging plasmids pMDLg/pRRE (for gag and pol expression), pMD.G (for expression of the VSV-G env protein), and pRSV.Rev (for rev expression) were co-transfected with the recombinant pRRL.cPPT.PGK.W.Sin-18 vectors to produce viral transduction units (TU). Virus supernatants were collected and filtered through a 0.2 μm filter and either used freshly, stored at −80° C. or ultracentrifuged to obtain high concentrations of viral stocks. Virus titers were determined by measuring the viral capsid protein p24 by ELISA. For in vitro transduction, PC12-αSyn were cultured directly in virus-containing media supplemented with 8 μg/ml polybrene.

Detection of Cell Death

Cell death will be assessed by quantifying intra- and extracellular lactate dehydrogenase (LDH). The ratio between the amount of LDH released and the amount remaining in the cells produces a measure of cell death.

$$Cell death ∝ \frac{extracellular LDH}{total LDH}$$

A sample of the medium will be the extracellular LDH sample. Cells were washed with PBS and extracted in cell lysis buffer (50 mM Tris pH 8.0, 150 mM NaCl, 5 mM EDTA, 1% Triton X-100, 10 μg/ml Aprotinin, 25 μg /ml Leupeptin, 10 μg/ml Pepstatin, 1 mM PMSF; all protease inhibitors purchased from Sigma Chemicals, St. Louis, Mo.). The cell homogenate were centrifuged at 4° C. for 3 minutes at 14,000 rpm. The supernatant were taken for intracellular LDH sample. LDH activity will be quantified using an LDH assay kit (Roche, Indianapolis, Ind.).

Apoptotic nuclei may be detected using TUNEL (ApopTag™, Serologicals Corporation, Norcross, Ga.). This technique tails nucleosome-sized DNA fragments with digoxigenin-dNTP followed by antibody conjugation. Nuclei may be counterstained with Hoechst 33342 (Molecular Probes, Eugene, Oreg.). Apoptosis may be quantified by a blinded assessor and expressed as the percentage of nuclei that are TUNEL-positive in two randomly selected 20× fields.

Immunocytochemistry

Routine indirect immunofluorescence was performed on 4% paraformaldehyde fixed VM culture. The fixed cells were incubated in a blocking solution consisting of 10% normal donkey serum (Jackson Immuno Research Laboratories Inc, West Grove, Pa.) and 0.1% Triton X-100 (Sigma, St. Louis, Mo.) in 0.1M phosphate buffered saline (PBS) for 1 hour at room temperature before transferring to the primary antibody solution. The primary antibodies were diluted in blocking solution and the cells were incubated overnight at 4° C. The primary antibodies used were raised against tyrosine hydroxylase (sheep anti-TH, 1:300, Pel-Freez Biologicals, Rogers, Ak.) and GIRK2 (rabbit; 1:80, Alomone Laboratories, Jerusalem, Israel). The cells were rinsed three times in 0.1 M PBS for five minutes each before the application of the secondary antibody solution for one hour at room temperature. The secondary antibodies were diluted in 10% normal donkey serum in 0.1M PBS. The secondary antibodies were conjugated to spectrally distinct fluorescent dyes to facilitate color separation (Alexa Fluor 488 conjugated donkey anti-sheep and Alexa Fluor 594 conjugated donkey anti-rabbit, Molecular Probes, Eugene, Oreg.). After rinsing in triplicate for ten minutes each in 0.1 M PBS, the cells were counterstained with 0.0005% Hoechst 33342 (Molecular Probes) in 0.1M Tris buffered saline. The coverslips containing the fixed cells were then rinsed in 0.1M PBS followed by distilled water and mounted onto slides using an aqueous mountant (Gel/Mount, Biømeda Corp., Calif.). Control coverslips immunostained without primary antibody were used to assess specificity of the technique.

Stereology

Design based stereology was performed by counters blinded to experimental groups on the stained coverslips using an integrated Axioskop 2 microscope (Carl Zeiss, Thornwood, N.Y.) and Stereoinvestigator image capture equipment and software (MicroBrightField, Williston, Vt.). A contour was drawn around each coverslip to identify the area of interest. A physical disector probe was utilized and counting frames were placed in a systematically random manner at approximately 125 sites per coverslip. The resultant coefficient of error for the stereological counts was used to assess precision (p<0.05).

Semiquantitative RT-PCR

One to three micrograms of amplified RNA was transcribed into cDNA with the SuperScript™ Preamplification Kit (Life Technologies) and random hexamers. The PCR reactions were carried out with 1×IN Reaction Buffer (Epicentre Technologies, Madison, Wis.), 1.4 nm of each primer, and 2.5 units of Taq I DNA polymerase (Promega, Madison, Wis.). Samples were amplified in an Eppendorf Thermocycler (Brinkmann Instruments, Westbury, N.Y.) under the following conditions: denaturing step at 95° C., 40 sec; annealing step at 55° C., 30 sec; amplification step at 72° C., 1 min for 20-30 cycles and a final amplification step at 72° C., 10 min. For semiquantitative RT-PCR, cDNA templates were normalized by β-actin-specific transcript and levels of gene transcription were detected by adjusting PCR cycling and primer design in such a way that each primer set amplified its corresponding gene product within a linear range, avoiding saturation of signals.

Quantitative Real Time PCR (Q-PCR)

RNA can be reverse transcribed to cDNA using Sensiscript (Qiagen, Valencia, Calif.) reverse transcriptase (RT) with oligo dT primer. RT reaction is performed at 37° C. for 1 hour and will be followed by inactivation of the enzyme at 93° C. for 5 min. Q-PCR may be performed using SYBR Green PCR master mix (PE Applied Biosystems, Foster City, Calif.). PCR mixture contains optimized concentrations of forward and reverse primers, cDNA and 1×SYBR Green PCR master mix. PCR amplification is performed as followed: 95° C. for 10 min, 49 cycles of 95° C. for 30 sec, 55° C. for 30 sec, 72° C. for 30 sec on a DNA Engine Opticon (MJ Research, Waltham, Mass.). Glyceraldehyde-3-phosphate dehydrogenase (GAPDH) and β-actin will be used as an internal control. The fold change in A9 and A10 cDNA relative to the internal controls is determined by: Fold change=2^{-ΔΔCt}, where
ΔΔCt=(Ct_{A9}−Ct_{internal control})−(Ct_{A10}−Ct_{internal control}).

TABLE 4
Genes Overexpressed by at Least 1.5 Fold in A9 Cells Relative to A10 Cells

| | Fold | | | |
|---|---|---|---|---|
| Gene ID | Induction | UniGene Name | UniGene Symbol | Representative mRNA Accession |
| 1418601_at | 5.89 | aldehyde dehydrogenase family 1, | Aldhla7 | NM_011921 // aldehyde |
| | | subfamily A7 | | dehydrogenase family 1, subfamily A7 |
| 1449571_at | 5.56 | thyrotropin releasing hormone receptor | Trhr | NM_013696 // thyrotropin releasing hormone receptor |
| 1437502_x_at | 4.07 | CD24a antigen | Cd24a | NM_009846 // CD24a antigen |
| 1448182_a_at | 3.92 | CD24a antigen | Cd24a | NM_009846 // CD24a antigen |
| 1423136_at | 3.57 | fibroblast growth factor 1 | Fgf1 | NM_010197 // fibroblast growth factor 1 |
| 1449494_at | 3.28 | RAB3C, member RAS oncogene family | Rab3c | NM_023852 // RAB3C, member RAS oncogene family |
| 1449348_at | 3.23 | membrane protein, palmitoylated 6 | Mpp6 | NM_019939 // membrane protein, |
| | | (MAGUK p55 subfamily member 6) | | palimitoylated 3 (MAGUK p55 subfamily member 6) |
| 1448954_at | 3.17 | RIKEN cDNA A330103B05 gene | A330103B05Rik | NM_020610 // RIKEN cDNA A330103B05 |
| 1449254_at | 2.92 | secreted phosphoprotein 1 | Spp1 | NM_009263 // secreted phosphoprotein 1 |
| 1452656_at | 2.90 | zinc finger, DHHC domain containing 2 | Zdhhc2 | NM_178395 // zinc finger, DHHC domain containing 2 |
| 1423287_at | 2.87 | cerebellin 1 precursor protein | Cbln1 | NM_019626 // cerebellin 1 precursor protein |
| 1449245_at | 2.84 | glutamate receptor, ionotropic, NMDA2C | Grin2c | NM_010350 // glutamate receptor, |
| | | (epsilon 3) | | ionotropic, NMDA2C (epsilon 3) |
| 143860_x_at | 2.74 | solute carrier family 25 (mitochondrial | Slc25a5 | NM_007451 // solute carrier family 25, member 5 |
| | | carrier; adenine nucleotide translocator), | | |
| | | member 5 | | |
| 1418104_at | 2.73 | RIKEN cDNA A330103B05 gene | A330103B05Rik | NM_020610 // RIKEN cDNA A330103B05 |
| 1422520_at | 2.73 | neurofilament 3, medium | Nef3 | NM_008691 // neurofilament 3, medium |
| 1448673_at | 2.70 | poliovirus receptor-related 3 | Pvrl3 | NM_021495 // poliovirus receptor-related 3 /// |
| | | | | NM_021496 // nectin-3beta /// NM_021497 // |
| | | | | nectin-3 gamma |
| 1421534_at | 2.65 | fibroblast growth factor inducible 15 | Fin15 | NM_008016 // fibroblast growth factor inducible 15 |
| 1438945_x_at | 2.56 | gap junction membrane channel protein | Gja1 | NM_010288 // gap junction membrane |
| | | alpha 1 | | channel protein alpha 1 |
| 1417071_s_at | 2.54 | cytochrome P450, family 4, subfamily v, | Cyp4v3 | NM_133969 // family 4 cytochrome P450 |
| | | polypeptide 3 | | |
| 1415800_at | 2.51 | gap junction membrane channel protein | Gja1 | NM_010288 // gap junction |
| | | alpha 1 | | membrane channel protein alpha 1 |
| 1436182_at | 2.50 | special AT-rich sequence binding protein 1 | Satb1 | NM_009122 // special |
| | | | | AT-rich sequence binding protein 1 |
| 1438922_x_at | 2.50 | solute carrier family 25 (mitochondrial | Slc25a5 | NM_007451 // solute carrier family 25, member 5 |
| | | carrier; adenine nucleotide translocator), | | |
| | | member 5 | | |
| 1452583_s_at | 2.49 | RIKEN cDNA A530057M15 gene | A530057M15Rik | NM_176963 // aldose 1-epimerase |
| 1427677_a_at | 2.45 | SRY-box containing gene 6 | Sox6 | NM_011445 // SRY-box containing gene 6 |
| 1437992_x_at | 2.45 | gap junction membrane channel protein | Gja1 | NM_010288 // gap junction |
| | | alpha 1 | | membrane channel protein alpha 1 |
| 1448600_s_at | 2.43 | vav 3 oncogene | Vav3 | NM_020505 // vav 3 oncogene /// |
| | | | | NM_146139 // vav 3 oncogene |
| 1438782_at | 2.42 | axonal-associated cell adhesion molecule | Axcam | NM_007518 // /// NM_173004 // |
| | | | | axonal-associated cell adhesion molecule |
| 1415861_at | 2.34 | tyrosinase-related protein 1 | Tyrp1 | NM_031202 // tyrosinase-related protein 1 |
| 1452142_at | 2.32 | gamma-aminobutyric acid (GABA-A) | Gabt1 | NM_178703 // gamma-aminobutyric |
| | | transporter 1 | | acid (GABA-A) transporter 1 |
| 1422600_at | 2.31 | RAS protein-specific guanine nucleotide- | Rasgrf1 | NM_011245 // RAS protein-specific |
| | | releasing factor 1 | | guanine nucleotide-releasing factor 1 |
| 1425846_a_at | 2.27 | calneuron 1 | Caln1 | NM_021371 // calneuron 1 |
| 1437401_at | 2.27 | insulin-like growth factor 1 | Igf1 | NM_010512 // insulin-like growth factor 1 /// |
| | | | | NM_184052 // insulin-like growth factor 1 |
| 1421349_x_at | 2.25 | BM88 antigen | Bm88-pending | NM_021316 // BM88 antigen |
| 1421841_at | 2.24 | fibroblast growth factor receptor 3 | Fgfr3 | NM_008010 // fibroblast growth factor receptor 3 |
| 1423286_at | 2.23 | cerebellin 1 precursor protein | Cbln1 | NM_019626 // cerebellin 1 precursor protein |
| 1434801_x_at | 2.15 | solute carrier family 25 (mitochondrial | Slc25a5 | NM_007451 // solute carrier family 25, member 5 |
| | | carrier; adenine nucleotide translocator), | | |
| | | member 5 | | |
| 1419225_at | 2.11 | calcium channel, voltage dependent, | Cacna2d3 | NM_009785 // calcium channel, |
| | | alpha2/delta subunit 3 | | voltage dependent, alpha2/delta subunit 3 |
| 1427019_at | 2.11 | protein tyrosine phosphatase, receptor | Ptprz1 | NM_011219 // protein tyrosine |
| | | type, Z polypeptide 1 | | phosphatase, receptor type, Z polypeptide 1 isoform 1 /// |
| | | | | phosphatase, receptor type, Z polypeptide 1 isoform 2 |
| 1450878_at | 2.11 | RIKEN cDNA 2210417O06 gene | 2210417O06Rik | NM_025618 // RIKEN cDNA 2210417O06 |
| 1438650_x_at | 2.08 | gap junction membrane channel protein | Gja1 | NM_010288 // gap junction |
| | | alpha 1 | | membrane channel protein alpha 1 |
| 1421180_at | 2.07 | limb expression 1 homolog (chicken) | Lix1 | NM_025681 // limb expression 1 homolog |
| 1416561_at | 2.07 | glutamic acid decarboxylase 1 | Gad1 | NM_008077 // glutamic acid decarboxylase 1 |
| 1417788_at | 2.05 | synuclein, gamma | Sncg | NM_011430 // synuclein, gamma |
| 1436027_at | 2.04 | oxysterol binding protein-like 11 | Osbpl11 | NM_176840 // oxysterol binding protein-like 11 |
| 1416007_at | 2.04 | special AT-rich sequence binding protein 1 | Satb1 | NM_009122 // special AT-rich |
| | | | | sequence binding protein 1 |
| 1434449_at | 2.03 | aquaporin 4 | Aqp4 | NM_009700 // aquaporin 4 |
| 1427919_at | 2.03 | RIKEN cDNA 1110039C07 gene | 1110039C07Rik | NM_026838 // sushi-repeat containing protein |
| 1432432_a_at | 2.03 | RAB3C, member RAS oncogene family | Rab3c | NM_023852 // RAB3C, member RAS oncogene family |
| 1422706_at | 2.03 | Mus musculus, clone IMAGE: 5370952, | NA | NA |
| | | mRNA | | |
| 1425542_a_at | 2.01 | protein phosphatase 2, regulatory subunit | Ppp2r5c | NM_012023 // gamma isoform of |
| | | B (B56), gamma isoform | | regulatory subunit B56, protein phosphatase 2A |
| 1435176_a_at | 2.00 | inhibitor of DNA binding 2 | Idb2 | NM_010496 // inhibitor of DNA binding 2 |
| 1439263_at | 2.00 | Mus musculus transcribed sequences | NA | NA |
| 1434893_at | 2.00 | Mus musculus, clone IMAGE: 1365759, | NA | NA |
| | | mRNA | | |
| 1419519_at | 1.98 | insulin-like growth factor 1 | Igf1 | NM_010512 // insulin-like growth factor 1 /// |
| | | | | NM_184052 // insulin-like growth factor 1 |
| 1436736_x_at | 1.98 | DNA segment, human D4S114 | D0H4S114 | NM_053078 // neuronal protein 3.1 |
| 1423854_a_at | 1.97 | RIKEN cDNA 1190017B18 gene | 1190017B18Rik | NA |
| 1423772_x_at | 1.97 | solute carrier family 25 (mitochondrial | Slc25a5 | NM_007451 // solute carrier family 25, member 5 |
| | | carrier; adenine nucleotide translocator), | | |
| | | member 5 | | |
| 1416183_a_at | 1.97 | lactate dehydrogenase 2, B chain | Ldh2 | NM_008492 // lactate dehydrogenase 2, B chain |
| 1425149_a_at | 1.95 | phosducin-like | Pdc1 | NM_026176 // phosducin-like |
| 1448955_s_at | 1.94 | Ca<2+>dependent activator protein for | Cadps | NM_012061 // Ca<2+>dependent |
| | | secretion | | activator protein for secretion |
| 1421348_a_at | 1.93 | BM88 antigen | Bm88-pending | NM_021316 // BM88 antigen |
| 1426283_at | 1.93 | RIKEN cDNA 6230410L23 gene | 6230410L23Rik | NA |
| 1416148_at | 1.93 | lysosomal-associated protein | Laptm4b | NM_033521 // lysosomal-associated |
| | | transmembrane 4B | | protein transmembrane 4B |
| 1448213_at | 1.93 | annexin A1 | Anxa1 | NM_010730 // annexin A1 |
| 1448334_a_at | 1.92 | cyclin I | Ccni | NM_017367 // cyclin I |
| 1448987_at | 1.92 | acetyl-Coenzyme A dehydrogenase, long- | Acad1 | NM_007381 // acetyl-Coenzyme |
| | | chain | | A dehydrogenase, long-chain |
| 1424988_at | 1.91 | myosin regulatory light chain interacting | Mir-pending | NM_153789 // myosin regulatory |
| | | protein | | light chain interacting protein |
| 1427186_a_at | 1.90 | myocyte enhancer factor 2A | Mef2a | NA |
| 1423288_s_at | 1.89 | cerebellin 1 precursor protein | Cbln1 | NM_019626 // cerebellin 1 precursor protein |
| 1416200_at | 1.89 | RIKEN cDNA 9230117N10 gene | 9230117N10Rik | NM_133775 // RIKEN cDNA 9230117N10 |
| 1456395_at | 1.89 | RIKEN cDNA A830037N07 gene | A830037N07Rik | NM_177113 // RIKEN cDNA A830037N07 gene |
| 1451190_a_at | 1.89 | SH3-binding kinase | Sbk-pending | NM_145587 // SH3-binding kinase |
| 1448729_a_at | 1.88 | septin 4 | 38234 | NM_011129 // peanut-like 2 homolog |
| 1450839_at | 1.88 | DNA segment, human D4S114 | D0H4S114 | NM_053078 // neuronal protein 3.1 |
| 1452284_at | 1.88 | protein tyrosine phosphatase, receptor | Ptprz1 | NM_011219 // protein tyrosine |
| | | type, Z polypeptide 1 | | phosphatase, receptor type, Z polypeptide 1 isoform 1 /// |
| | | | | NM_178180 // protein tyrosine phosphatase, |
| | | | | receptor type, Z polypeptide 1 isoform 2 |
| 1424034_at | 1.87 | RAR-related orphan receptor alpha | Rora | NM_013646 // RAR-related orphan receptor alpha |
| 1416749_at | 1.85 | protease, serine, 11 (Igf binding) | Prss11 | NM_019564 // protease, serine, 11 (Igf binding) |
| 1434229_a_at | 1.85 | polymerase (DNA directed), beta | Polb | NM_011130 // polymerase (DNA directed), beta |
| 1434877_at | 1.85 | neuronal pentraxin 1 | Nptx1 | NM_008730 // neuronal pentraxin 1 |
| 1421129_a_at | 1.85 | ATPase, Ca++ transporting, ubiquitous | Atp2a3 | NM_016745 // ATPase, Ca++ transporting, ubiquitous |
| 1418929_at | 1.83 | estrogen-related receptor beta like 1 | Esrrbl1 | NM_028680 // huntingtin- |
| | | | | interacting protein-1 protein interactor |
| 1427344_s_at | 1.83 | RIKEN cDNA 4930526B11 gene | 4930526B11Rik | NA |
| 1416114_at | 1.82 | SPARC-like 1 (mast9, hevin) | Sparcl1 | NM_010097 // SPARC-like 1 (mast9, hevin) |
| 1438545_at | 1.82 | solute carrier family 25 (mitochondrial | Slc25a5 | NM_007451 // solute carrier family 25, member 5 |
| | | carrier; adenine nucleotide translocator), | | |
| | | member 5 | | |
| 1437874_s_at | 1.81 | hexosaminidase B | Hexb | NM_010422 // hexosaminidase B |
| 1452123_s_at | 1.81 | RIKEN cDNA 6030440G05 gene | 6030440G05Rik | NM_145148 // GRP1-binding protein GRSP1 |
| 1451313_a_at | 1.81 | RIKEN cDNA 1110067D22 gene | 1110067D22Rik | NM_173752 // RIKEN cDNA 1110067D22 |
| 1424847_at | 1.80 | neurofilament, heavy polypeptide | Nefh | NA |
| 1449312_at | 1.80 | neuropeptide Y receptor Y5 | Npy5r | NM_016708 // neuropeptide Y receptor Y5 |
| 1448527_at | 1.80 | programmed cell death 10 | Pdcd10 | NM_019745 // programmed cell death 10 |
| 1438292_x_at | 1.80 | adenosine kinase | Adk | NM_007411 // /// NM_134079 // adenosine kinase |
| 1451236_at | 1.79 | RAS-like, estrogen-regulated, growth- | Rerg | NM_181988 // RAS-like, |
| | | inhibitor | | estrogen-regulated, growth-inhibitor |
| 1455239_at | 1.79 | Mus musculus, Similar to RIKEN cDNA | NA | NA |
| | | 6330512M04 gene, clone MGC: 58491 | | |
| | | IMAGE: 6534368, mRNA, complete cds | | |
| 1452090_a_at | 1.78 | olfactomedin 3 | Olfm3 | NM_153157 // olfactomedin 3, |
| | | | | isoformB /// NM_153458 // olfactomedin 3, isoform A |
| 1424095_at | 1.78 | RIKEN cDNA 2310009A18 gene | 2310009A18Rik | NM_025517 // RIKEN cDNA 2310009A18 |
| 1423312_at | 1.75 | trophoblast glycoprotein | Tpbg | NM_011627 // trophoblast glycoprotein |
| 1422824_s_at | 1.74 | epidermal growth factor receptor pathway | Eps8 | NM_007945 // epidermal growth factor |
| | | substrate 8 | | receptor pathway substrate 8 |
| 1451991_at | 1.73 | Eph receptor A7 | Epha7 | NM_010141 // Eph receptor A7 |
| 1452283_at | 1.73 | expressed sequence AW123240 | AW123240 | NA |
| 1453060_at | 1.72 | RIKEN cDNA 6530413N01 gene | 6530413N01Rik | NM_026380 // RIKEN cDNA 6530413N01 |
| 1450940_at | 1.72 | ganglioside-induced differentiation- | Gdap1 | NM_010267 // ganglioside-induced |
| | | associated-protein 1 | | differentiation-associated-protein 1 |
| 1418758_a_at | 1.72 | pleckstrin homology, Sec7 and coiled-coil | Pscd3 | NM_011182 // pleckstrin homology, |
| | | domains 3 | | Sec7 and coiled-coil domains 3 |
| 1417318_at | 1.71 | deleted in bladder cancer chromosome | Dbccr1 | NM_019967 // deleted in bladder cancer |
| | | region candidate 1 (human) | | chromosome region candidate 1 |
| 1415908_at | 1.71 | testis-specific protein, Y-encoded-like | Tspy1 | NM_009433 // testis-specific protein, Y-encoded-like |
| 1427004_at | 1.71 | F-box only protein 2 | Fbxo2 | NM_176848 // F-box only protein 2 |
| 1449929_at | 1.70 | t-complex-associated-testis-expressed 1- | Tcte11 | NM_025975 // t-complex- |
| | | like | | associated-testis-expressed 1-like |
| 1429318_a_at | 1.70 | quaking | Qk | NM_021881 // quaking protein |
| 1421878_at | 1.70 | mitogen activated protein kinase 9 | Mapk9 | NM_016961 // mitogen activated protein kinase 9 |
| 1455534_s_at | 1.70 | oxysterol binding protein-like 11 | Osbpl11 | NM_176840 // oxysterol binding protein-like 11 |
| 1459903_at | 1.70 | RIKEN cDNA 2900057C09 gene | 2900057C09Rik | NA |
| 1434109_at | 1.69 | RIKEN cDNA A930014C21 gene | A930014C21Rik | NM_172507 // RIKEN cDNA A930014C21 |
| 1422823_at | 1.69 | epidermal growth factor receptor pathway | Eps8 | NM_007945 // epidermal growth factor |
| | | substrate 8 | | receptor pathway substrate 8 |
| 1430326_s_at | 1.69 | low molecular mass ubiquinone-binding | Qpc-pending | NM_025352 // low molecular |
| | | protein | | mass ubiquinone-binding protein |
| 1455235_x_at | 1.68 | lactate dehydrogenase 2, B chain | Ldh2 | NM_008492 // lactate dehydrogenase 2, B chain |
| 1417403_at | 1.68 | ELOVL family member 6, elongation of | Elov16 | NM_130450 // ELOVL family |
| | | long chain fatty acids (yeast) | | member 6, elongation of long chain fatty acids |
| 1415930_a_at | 1.68 | microtubule-associated protein 1 light | Map1lc3 | NM_026160 // microtubule- |
| | | chain 3 | | associated protein 1 light chain 3 |
| 1416958_at | 1.68 | nuclear receptor subfamily 1, group D, | Nr1d2 | NM_011584 // nuclear receptor |
| | | member 2 | | subfamily 1, group D, member 2 |
| 1415783_at | 1.68 | vacuolar protein sorting 35 | Vps35 | NM_008582 // /// |
| | | | | NM_022997 // vacuolar protein sorting 35 |
| 1424367_a_at | 1.68 | homer homolog 2 (Drosopila) | Homer2 | NA |
| 1428187_at | 1.68 | RIKEN cDNA 9130415E20 gene | 9130415E20Rik | NM_175169 // RIKEN cDNA 9130415E20 |
| 1449921_s_at | 1.68 | copine Vl | Cpne6 | NM_009947 // copine VI |
| 1438606_a_at | 1.67 | DNA segment, Jeremy M. Boss 3 | D0Jmb3 | NA |
| 1423907_a_at | 1.67 | NADH dehydrogenase (ubiquinone) Fe—S | Ndufs8 | NM_144870 // NADH dehydrogenase |
| | | protein 8 | | (ubiquinone) Fe—S protein 8 |
| 1436915_x_at | 1.67 | lysosomal-associated protein | Laptm4b | NM_033521 // lysosomal- |
| | | transmembrane 4B | | associated protein transmembrane 4B |
| 1422052_at | 1.67 | cadherin 8 | Cdh8 | NM_007667 // cadherin 8 |
| 1456005_a_at | 1.67 | BCL2-like 11 (apoptosis facilitator) | Bcl2l11 | NM_009754 // BCL2-like 11 apoptosis facilitator |
| 1423135_at | 1.67 | thymus cell antigen 1, theta | Thy1 | NM_009382 // thymus cell antigen 1, theta |
| 1439255_s_at | 1.67 | transmembrane 7 superfamily member 1 | Tm7sf1 | NM_031999 // transmembrane 7 superfamily member 1 |
| 1418506_a_at | 1.66 | peroxiredoxin 2 | Prdx2 | NM_011563 // peroxiredoxin 2 |
| 1452130_at | 1.66 | RIKEN cDNA 2310042M24 gene | 2310042M24Rik | NM_025868 // RIKEN cDNA 2310042M24 |
| 1423796_at | 1.66 | splicing factor proline/glutamine rich | Sfpq | NM_023603 // splicing factor |
| | | (polypyrimidine tract binding protein | | proline/glutamine rich (polypyrimidine tract binding |
| | | associated) | | protein associated) |
| 1436912_at | 1.66 | RIKEN cDNA 3110038O15 gene | 3110038O15Rik | NA |
| 1417151_a_at | 1.65 | neurotensin receptor 2 | Ntsr2 | NM_008747 // neurotensin receptor 2 |
| 1460192_at | 1.65 | oxysterol binding protein-like 1A | Osbp11a | NM_020573 // oxysterol binding protein-like 1A |
| 1438957_x_at | 1.65 | CDP-diacylglycerol synthase | Cds2 | NM_138651 // phosphatidate cytidylyltransferase 2 |
| | | (phosphatidate cytidylyltransferase) 2 | | |
| 1434099_at | 1.65 | caspase 7 | Casp7 | NM_007611 // caspase 7 |
| 1434256_s_at | 1.65 | CDP-diacylglycerol synthase | Cds2 | NM_138651 // phosphatidate cytidylyltransferase 2 |
| | | (phosphatidate cytidylyltransferase) 2 | | |
| 1435614_s_at | 1.65 | RAS protein-specific guanine nucleotide- | Rasgrf1 | NM_011245 // RAS protein-specific |
| | | releasing factor 1 | | guanine nucleotide-releasing factor 1 |
| 1449256_a_at | 1.65 | RAB11a, member RAS oncogene family | Rab11a | NM_017382 // RAB11a, member RAS oncogene family |
| 1419244_a_at | 1.65 | RAB14, member RAS oncogene family | Rab14 | NM_026697 // RAB14, member RAS oncogene family |
| 1448210_at | 1.64 | RAB1, member RAS oncogene family | Rab1 | NM_008996 // RAB1, member RAS oncogene family |
| 1418073_at | 1.64 | acyl-Coenzyme A thioesterase 2, | Acate2-pending | NM_019736 // acyl-Coenzyme |
| | | mitochondrial | | A thioesterase 2, mitochondrial |
| 1436934_s_at | 1.64 | aconitase 2, mitochondrial | Aco2 | NM_080633 // aconitase 2, mitochondrial |
| 1433562_s_at | 1.64 | ATP synthase, H+ transporting, | Atp5f1 | NM_009725 // ATP synthase, |
| | | mitochondrial F0 complex, subunit b, | | H+ transporting, mitochondrial |
| | | isoform 1 | | F0 complex, subunit b, isoform 1 |
| 1426282_at | 1.64 | neurotrimin | Hnt-pending | NM_172290 // neurotrimin |
| 1455422_x_at | 1.64 | septin4 | 38234 | NM_011129 // peanut-like 2 homolog |
| 1452141_a_at | 1.63 | selenoprotein P, plasma, 1 | Sepp1 | NM_009155 // selenoprotein P, plasma, 1 |
| 1437164_x_at | 1.63 | ATP synthase, H+ transporting, | Atp5o | NM_138597 // ATP synthase, |
| | | mitochondrial F1 complex, O subunit | | H+ transporting, mitochondrial F1 complex, O subunit |
| 1428107_at | 1.62 | SH3-binding domain glutamic acid-rich | Sh3bgrl | NM_019989 // SH3-binding domain |
| | | protein like | | glutamic acid-rich protein like |
| 1426917_s_at | 1.62 | RIKEN cDNA 4833415E20 gene | 4833415E20Rik | NA |
| 1423283_at | 1.61 | phosphatidylinositol transfer protein | Pitpn | NM_008850 // phosphatidylinositol transfer protein |
| 1423079_a_at | 1.61 | translocase of outer mitochondrial | Tomm20-pending | NM_024214 // translocase of |
| | | membrane 20 homolog (yeast) | | outer mitochondrial membrane 20 homolog |
| 1423172_at | 1.61 | N-ethylmaleimide sensitive fusion protein | Napb | NM_019632 // N-ethylmaleimide sensitive |
| | | attachment protein beta | | fusion protein attachment protein beta |
| 1416127_a_at | 1.61 | aspartyl aminopeptidase | Dnpep | NM_016878 // aspartyl aminopeptidase |
| 1452347_at | 1.61 | myocyte enhancer factor 2A | Mef2a | NA |
| 1424573_at | 1.61 | RIKEN cDNA 3110020O18 gene | 3110020O18Rik | NM_028445 // /// NM_028876 // CGI-100-like protein |
| 1448237_x_at | 1.61 | lactate dehydrogenase 2, B chain | Ldh2 | NM_008492 // lactate dehydrogenase 2, B chain |
| 1436750_a_at | 1.60 | 3-oxoacid CoA transferase | Oxct | NM_024188 // 3-oxoacid CoA transferase |
| 1417340_at | 1.60 | thioredoxin-like 2 | Txnl2 | NM_023140 // thioredoxin-like 2 |
| 1424488_a_at | 1.60 | inorganic pyrophosphatase 2 | Sid6306-pending | NM_146141 // inorganic pyrophosphatase 2 |
| 1416904_at | 1.60 | muscleblind-like (Drosophila) | Mbn1 | NM_020007 // muscleblind-like |
| 1415753_at | 1.60 | cDNA sequence BC005632 | BC005632 | NM_145421 // cDNA sequence BC005632 |
| 1417404_at | 1.60 | ELOVL family member 6, elongation of | Elov16 | NM_130450 // ELOVL family member 6, |
| | | long chain fatty acids (yeast) | | elongation of long chain fatty acids |
| 1426858_at | 1.60 | inhibin beta-B | Inhbb | NA |
| 1423399_a_at | 1.59 | YY1 associated factor 2 | Yaf2 | NM_024189 // YY1 associated factor 2 |
| 1448474_at | 1.59 | NIMA (never in mitosis gene a)-related | Nek7 | NM_021605 // NIMA (never in |
| | | expressed kinase 7 | | mitosis gene a)-related expressed kinase 7 |
| 1419872_at | 1.59 | colony stimulating factor 1 receptor | Csflr | NM_007779 // colony stimulating factor 1 receptor |
| 1418937_at | 1.59 | deiodinase, iodothyronine, type II | Dio2 | NM_010050 // deiodinase, iodothyronine, type II |
| 1420161_at | 1.58 | Mus musculus transcribed sequences | NA | NA |
| 1424293_s_at | 1.58 | RIKEN cDNA 2610319K07 gene | 2610319K07Rik | NM_028264 // RIKEN cDNA 2610319K07 |
| 1428788_at | 1.57 | RIKEN cDNA 1700012G19 gene | 1700012G19Rik | NM_025954 // RIKEN cDNA 1700012G19 |
| 1421022_x_at | 1.57 | acylphosphatase 1, erythrocyte (common) | Acyp1 | NM_025421 // |
| | | type | | acylphosphatase 1, erythrocyte (common) type |
| 1422640_at | 1.57 | protocadherin beta 9 | Pcdhb9 | NM_053134 // protocadherin beta 9 |
| 1416501_at | 1.57 | 3-phosphoinositide dependent protein | Pdpk1 | NM_011062 // 3-phosphoinositide |
| | | kinase-1 | | dependent protein kinase-1 |
| 1426312_at | 1.57 | brain and reproductive organ-expressed | Bre | NM_144541 // brain and reproductive |
| | | protein | | organ-expressed protein |
| 1419554_at | 1.57 | RIKEN cDNA B430305P08 gene | B430305P08Rik | NA |
| 1460180_at | 1.56 | hexosaminidase B | Hexb | NM_010422 // hexosaminidase B |
| 1451542_at | 1.56 | single-stranded DNA binding protein 2 | Ssbp2 | NM_024186 // single-stranded DNA binding |
| | | | | protein 2 /// NM_024272 // single-stranded |
| | | | | DNA binding protein 2 |
| 1418072_at | 1.56 | histone 1, H2bc | Hist1h2bc | NM_023422 // histone 1, H2bc |
| 1452152_at | 1.56 | expressed sequence AI642036 | AI642036 | NA |
| 1416149_at | 1.56 | oligodendrocyte transcription factor 1 | Olig1 | NM_016968 // oligodendrocyte transcription factor 1 |
| 1423515_at | 1.56 | sodium channel, voltage-gated, type VIII, | Scn8a | NM_011323 // sodium channel, |
| | | alpha polypeptide | | voltage-gated, type VIII, alpha polypeptide |
| 1418501_a_at | 1.56 | oxidation resistance 1 | Oxr1 | NM_130885 // oxidation resistance 1 |
| 1451235_at | 1.56 | BM88 antigen | Bm88-pending | NM_021316 // BM88 antigen |
| 1423978_at | 1.55 | SH3-binding kinase | Sbk-pending | NM_145587 // SH3-binding kinase |
| 1450757_at | 1.55 | cadherin 11 | Cdh11 | NM_009866 // cadherin 11 |
| 1450888_at | 1.55 | brain protein 14 | Brp14 | NA |
| 1436824_x_at | 1.55 | ring finger protein 26 | Rnf26 | NM_153762 // ring finger protein 26 |
| 1427182_s_at | 1.55 | DNA segment, Chr 18, ERATO Doi 653, | D18Ertd653e | NM_172631 // DNA segment, |
| | | expressed | | Chr 18, ERATO Doi 653, expressed |
| 1434100_x_at | 1.55 | caspase 7 | Casp7 | NM_007611 // caspase 7 |
| 1434261_at | 1.55 | hypothetical protein LOC244668 | LOC244668 | NA |
| 1425580_a_at | 1.55 | RIKEN cDNA 5330434F23 gene | 5330434F23Rik | NM_181414 // RIKEN cDNA 5330434F23 gene |
| 1425562_s_at | 1.55 | tRNA nucleotidyl transferase, CCA- | Trnt1 | NM_027296 // tRNA nucleotidyl |
| | | adding, 1 | | transferase, CCA-adding, 1 |
| 1416202_at | 1.54 | B-cell receptor-associated protein 37 | Bcap37 | NM_007531 // B-cell receptor-associated protein 37 |
| 1452202_at | 1.54 | phosphodiesterase 2A, cGMP-stimulated | Pde2a | NA |
| 1449815_a_at | 1.54 | single-stranded DNA binding protein 2 | Ssbp2 | NM_024186 // single-stranded DNA binding |
| | | | | protein 2 /// NM_024272 // single-stranded DNA binding |
| | | | | protein 2 |
| 1422578_at | 1.54 | citrate synthase | Cs | NM_026444 // citrate synthase |
| 1421396_at | 1.54 | proprotein convertase subtilisin/kexin type 1 | Pcsk1 | NM_013628 // proprotein |
| 1427288_at | 1.54 | amyloid beta (A4) precursor protein- | Apba2 | NA |
| | | binding, family A, member 2 | | convertase subtilisin/kexin type 1 |
| 1423113_a_at | 1.54 | RIKEN cDNA 1100001F19 gene | 1100001F19Rik | NM_025356 // RIKEN cDNA 1100001F19 |
| 1456542_s_at | 1.53 | Mus musculus RIKEN cDNA 2700038P16 | NA | NA |
| | | gene, mRNA (cDNA clone | | |
| | | IMAGE: 4923133), partial cds | | |
| 1425575_at | 1.53 | Eph receptor A3 | Epha3 | NM_010140 // Eph receptor A3 |
| 1427470_s_at | 1.53 | N-ethylmaleimide sensitive fusion protein | Napb | NM_019632 // N-ethylmaleimide sensitive |
| | | attachment protein beta | | fusion protein attachment protein beta |
| 1423784_at | 1.53 | glycyl-tRNA synthetase | Gars | NM_180678 // glycyl-tRNA synthetase |
| 1424885_at | 1.53 | RIKEN cDNA A630065K24 gene | A630065K24Rik | NM_144810 // RIKEN cDNA A630065K24 |
| 1422504_at | 1.53 | glycine receptor, beta subunit | Glrb | NM_010298 // glycine receptor, beta subunit |
| 1451290_at | 1.53 | RIKEN cDNA 1010001H21 gene | 1010001H21Rik | NM_025735 // RIKEN cDNA 4922501H04 |
| 1415918_a_at | 1.53 | triosephosphate isomerase | Tpi | NM_009415 // triosephosphate isomerase |
| 1429240_at | 1.53 | StAR-related lipid transfer (START) | Stard4 | NM_133774 // StAR-related lipid transfer |
| | | domain containing 4 | | (START) domain containing 4 |
| 1455893_at | 1.52 | RIKEN cDNA 2610028F08 gene | 2610028F08Rik | NM_172815 // RIKEN cDNA 2610028F08 |
| 1435659_a_at | 1.52 | triosephosphate isomerase | Tpi | NM_009415 // triosephosphate isomerase |
| 1424211_at | 1.52 | RIKEN cDNA 5730438N18 gene | 5730438N18Rik | NM_027460 // RIKEN cDNA 5730438N18 |
| 1424842_a_at | 1.52 | RIKEN cDNA 0610025G21 gene | 0610025G21Rik | NM_029270 // RIKEN cDNA 0610025G21 /// |
| | | | | NM_146161 // RIKEN cDNA 0610025G21 |
| 1417900_a_at | 1.52 | very low density lipoprotein receptor | Vldlr | NM_013703 // very low density lipoprotein receptor |
| 1427069_at | 1.51 | DNA segment, Chr 1, ERATO Doi 578, | D1Ertd578e | NM_175127 // DNA segment, Chr 1, ERATO |
| | | expressed | | Doi 578, expressed |
| 1435970_at | 1.51 | nemo like kinase | Nlk | NM_008702 // nemo like kinase |
| 1416989_at | 1.51 | RIKEN cDNA 3100002B05 gene | 3100002B05Rik | NM_026664 // RIKEN cDNA 3100002B05 |
| 1451695_a_at | 1.51 | glutathione peroxidase 4 | Gpx4 | NM_008162 // glutathione peroxidase 4 |
| 1423736_a_at | 1.51 | RIKEN cDNA 4933427L07 gene | 4933427L07Rik | NM_026986 // /// NM_027727 // RIKEN |
| | | | | cDNA 4933427L07 |
| 1420570_x_at | 1.51 | T-cell leukemia/lymphoma 1B, 3 | Tcl1b3 | NM_013772 // T-cell leukemia/lymphoma 1B, 3 |
| 1421479_at | 1.51 | zinc finger protein 318 | Zfp318 | NM_021346 // zinc finger protein 318 |
| 1426994_at | 1.51 | expressed sequence AI836256 | AI836256 | NA |
| 1417283_at | 1.51 | Ly6/neurotoxin 1 | Lynx1 | NM_011838 // Ly6/neurotoxin 1 |
| 1448259_at | 1.51 | follistatin-like | Fstl | NM_008047 // follistatin-like |
| 1451425_a_at | 1.51 | makorin, ring finger protein, 1 | Mkrn1 | NM_018810 // makorin, ring finger protein, 1 |
| 1424100_s_at | 1.51 | BM88 antigen | Bm88-pending | NM_021316 // BM88 antigen |
| 1431074_a_at | 1.50 | RIKEN cDNA 1110020B03 gene | 1110020B03Rik | NM_145823 // retinal degeneration B beta |
| 1451269_at | 1.50 | RIKEN cDNA 2700099C19 gene | 2700099C19Rik | NM_028303 // RIKEN cDNA 2700099C19 |
| 1431619_a_at | 1.50 | dystrobrevin binding protein 1 | Dtnbp1 | NM_025772 // dystrobrevin binding protein 1 |
| 1417892_a_at | 1.50 | sirtuin 3 (silent mating type information | Sirt3 | NM_022433 // sirtuin 3 (silent mating type information |
| | | regulation 2, homolog) 3 (S. cerevisiae) | | regulation 2, homolog) 3 |

TABLE 5
Genes Overexpressed by at Least 1.5 Fold in A10 Cells Relative to A9 Cells

| | Fold | | UniGene | |
|---|---|---|---|---|
| ID | Induction | UniGene Name | Symbol | Representative mRNA Accession |
| 1415904_at | −17.94 | lipoprotein lipase | Lpl | NM_008509 // lipoprotein lipase |
| 1424525_at | −16.19 | gastrin releasing peptide | Grp | NM_175012 // gastrin releasing peptide |
| 1422825_at | −6.96 | cocaine and amphetamine regulated transcript | Cart-pending | NM_013732 // cocaine and amphetamine |
| | | | | regulated transcript |
| 1425926_a_at | −6.25 | orthodenticle homolog 2 (Drosophila) | Otx2 | NM_144841 // orthodenticle homolog 2 |
| 1437405_a_at | −5.06 | insulin-like growth factor binding protein 4 | Igfbp4 | NM_010517 // insulin-like growth factor |
| | | | | binding protein 4 |
| 1452004_at | −5.05 | calcitonin/calcitonin-related polypeptide, alpha | Calca | NM_007587 // calcitonin/calcitonin-related |
| | | | | polypeptide, alpha |
| 1423756_s_at | −4.95 | insulin-like growth factor binding protein 4 | Igfbp4 | NM_010517 // insulin-like growth factor |
| | | | | binding protein 4 |
| 1418047_at | −4.71 | neurogenic differentiation 6 | Neurod6 | NM_009717 // neurogenic differentiation 6 |
| 1449360_at | −4.71 | colony stimulating factor 2 receptor, beta 2, | Csf2rb2 | NM_007781 // colony stimulating factor 2 |
| | | low-affinity (granulocyte-macrophage) | | receptor, beta 2, low-affinity (granulocyte- |
| | | | | macrophage) |
| 1456307_s_at | −4.12 | adenylate cyclase 7 | Adcy7 | NM_007406 // adenylate cyclase 7 |
| 1417065_at | −4.07 | early growth response 1 | Egr1 | NM_007913 // early growth response 1 |
| 1426528_at | −3.85 | neuropilin 2 | Nrp2 | NM_010939 // neuropilin 2 |
| 1417962_s_at | −3.77 | growth hormone receptor | Ghr | NM_010284 // growth hormone receptor |
| 1428664_at | −3.56 | vasoactive intestinal polypeptide | Vip | NM_011702 // vasoactive intestinal |
| | | | | polypeptide |
| 1427509_at | −3.54 | RIKEN cDNA 0610007P22 gene | 0610007P22Rik | NM_026676 // RIKEN cDNA 0610007P22 |
| 1417680_at | −3.52 | potassium voltage-gated channel, shaker-related | Kcna5 | NM_008419 // potassium voltage-gated |
| | | subfamily, member 5 | | channel, shaker-related subfamily, member 5 |
| 1437029_at | −3.43 | tachykinin receptor 3 | Tacr3 | NM_021382 // neurokinin-3 receptor |
| 1417343_at | −3.37 | FXYD domain-containing ion transport regulator 6 | Fxyd6 | NM_022004 // FXYD domain-containing ion |
| | | | | transport regulator 6 |
| 1428301_at | −3.36 | RIKEN cDNA 2610042L04 gene | 2610042L04Rik | NM_025940 // RIKEN cDNA 2610042L04 |
| 1452731_x_at | −3.34 | RIKEN cDNA 2610042L04 gene | 2610042L04Rik | NM_025940 // RIKEN cDNA 2610042L04 |
| 1417090_at | −3.18 | reticulocalbin | Rcn | NM_009037 // reticulocalbin |
| 1418599_at | −3.17 | procollagen, type XI, alpha 1 | Col11a1 | NM_007729 // procollagen, type XI, alpha 1 |
| 1417504_at | −3.14 | calbindin-28K | Calb1 | NM_009788 // calbindin-28K |
| 1436862_at | −3.01 | double C2, alpha | Doc2a | NM_010069 // double C2, alpha |
| 1417376_a_at | −2.95 | immunoglobulin superfamily, member 4 | Igsf4 | NM_018770 // immunoglobulin superfamily, |
| | | | | member 4 |
| 1423100_at | −2.95 | FBJ osteosarcoma oncogene | Fos | NM_010234 // FBJ osteosarcoma oncogene |
| 1418084_at | −2.94 | neuropilin | Nrp | NM_008737 // neuropilin |
| 1420465_s_at | −2.93 | major urinary protein 1 | Mup1 | NM_031188 // major urinary protein 1 |
| 1448738_at | −2.88 | calbindin-28K | Calb1 | NM_009788 // calbindin-28K |
| 1426301_at | −2.85 | activated leukocyte cell adhesion molecule | Alcam | NM_009655 // activated leukocyte cell |
| | | | | adhesion molecule |
| 1435627_x_at | −2.73 | MARCKS-like protein | Mlp | NM_010807 // MARCKS-like protein |
| 1437226_x_at | −2.72 | MARCKS-like protein | Mlp | NM_010807 // MARCKS-like protein |
| 1425918_at | −2.71 | Mus musculus, Similar to EGL nine homolog 3 | NA | NA |
| | | (C. elegans), clone MGC: 36685 IMAGE: 5371854, | | |
| | | mRNA, complete cds | | |
| 1423427_at | −2.69 | adenylate cyclase activating polypeptide 1 | Adcyap1 | NM_009625 // adenylate cyclase activating |
| | | | | polypeptide 1 |
| 1428379_at | −2.67 | solute carrier family 17 (sodium-dependent inorganic | Slc17a6 | NM_080853 // solute carrier family 17 |
| | | phosphate cotransporter), member 6 | | (sodium-dependent inorganic phosphate |
| | | | | cotransporter), member 6 |
| 1424586_at | −2.61 | DNA sequence AF424697 | AF424697 | NM_153078 // DNA sequence AF424697 |
| 1421595_at | −2.58 | NA | NA | NA |
| 1449240_at | −2.58 | G substrate | Gsbs-pending | NM_011153 // G substrate |
| 1419406_a_at | −2.57 | B-cell CLL/lymphoma 11A (zinc finger protein) | Bcl11a | NM_016707 // B-cell CLL/lymphoma 11A |
| | | | | (zinc finger protein) |
| 1426208_x_at | −2.56 | pleiomorphic adenoma gene-like 1 | Plagl1 | NM_009538 // pleiomorphic adenoma gene- |
| | | | | like 1 |
| 1435415_x_at | −2.55 | MARCKS-like protein | Mlp | NM_010807 // MARCKS-like protein |
| 1451129_at | −2.55 | calbindin 2 | Calb2 | NM_007586 // calbindin 2 |
| 1460716_a_at | −2.52 | core binding factor beta | Cbfb | NM_022309 // core binding factor beta |
| 1417377_at | −2.47 | immunoglobulin superfamily, member 4 | Igsf4 | NM_018770 // immunoglobulin superfamily, |
| | | | | member 4 |
| 1419077_at | −2.47 | membrane protein, palmitoylated 3 (MAGUK p55 | Mpp3 | NM_007863 // Mpp3 membrane protein, |
| | | subfamily member 3) | | palmitoylated 3 |
| 1450047_at | −2.43 | heparan sulfate 6-O-sulfotransferase 2 | Hs6st2 | NM_015819 // heparan sulfate 6-O- |
| | | | | sulfotransferase 2 |
| 1418815_at | −2.42 | cadherin 2 | Cdh2 | NM_007664 // cadherin 2 |
| 1423760_at | −2.41 | CD44 antigen | Cd44 | NA |
| 1456108_x_at | −2.41 | zinc finger protein 179 | Zfp179 | NM_009548 // zinc finger protein 179 |
| 1448468_a_at | −2.40 | potassium voltage-gated channel, shaker-related | Kcnab1 | NM_010597 // potassium voltage-gated |
| | | subfamily, beta member 1 | | channel, shaker-related subfamily, beta |
| | | | | member 1 |
| 1419473_a_at | −2.38 | cholecystokinin | Cck | NM_031161 // cholecystokinin |
| 1452406_x_at | −2.37 | erythroid differentiation regulator | edr | NM_133362 // erythroid differentiation |
| | | | | regulator |
| 1423213_at | −2.36 | plexin C1 | Plxnc1 | NM_018797 // plexin C1 |
| 1425603_at | −2.29 | RIKEN cDNA 0610011I04 gene | 0610011I04Rik | NM_025326 // RIKEN cDNA 0610011I04 |
| 1421508_at | −2.28 | odd Oz/ten-m homolog 1 (Drosophila) | Odz1 | NM_011855 // odd Oz/ten-m homolog 1 |
| 1448943_at | −2.23 | neuropilin | Nrp | NM_008737 // neuropilin |
| 1427917_s_at | −2.22 | single-stranded DNA binding protein 3 | Ssbp3 | NM_023672 // single-stranded DNA-binding |
| | | | | protein |
| 1418304_at | −2.21 | photoreceptor cadherin | Prcad-pending | NM_130878 // photoreceptor cadherin |
| 1448553_at | −2.18 | myosin, heavy polypeptide 7, cardiac muscle, beta | Myh7 | NM_080728 // myosin, heavy polypeptide 7, |
| | | | | cardiac muscle, beta |
| 1417378_at | −2.14 | immunoglobulin superfamily, member 4 | Igsf4 | NM_018770 // immunoglobulin superfamily, |
| | | | | member 4 |
| 1418835_at | −2.14 | pleckstrin homology-like domain, family A, member 1 | Phlda1 | NM_009344 // pleckstrin homology-like |
| | | | | domain, family A, member 1 |
| 1415716_a_at | −2.12 | ribosomal protein S27 | Rps27 | NM_027015 // ribosomal protein S27 |
| 1418734_at | −2.09 | histocompatibility 2, Q region locus 1 | H2-Q1 | NM_010390 // histocompatibility 2, Q region |
| | | | | locus 1 |
| 1426951_at | −2.08 | cysteine-rich motor neuron 1 | Crim1 | NA |
| 1426002_a_at | −2.08 | cell division cycle 7 (S. cerevisiae) | Cdc7 | NM_009863 // cell division cycle 7 |
| 1450123_at | −2.07 | ryanodine receptor 2, cardiac | Ryr2 | NM_023868 // ryanodine receptor 2, cardiac |
| 1452035_at | −2.05 | procollagen, type IV, alpha 1 | Col4a1 | NM_009931 // procollagen, type IV, alpha 1 |
| 1418603_at | −2.05 | arginine vasopressin receptor 1A | Avpr1a | NM_016847 // arginine vasopressin receptor |
| | | | | 1A |
| 1417765_a_at | −2.04 | amylase 1, salivary | Amy1 | NM_007446 // amylase 1, salivary |
| 1416181_at | −2.03 | mesoderm development candiate 2 | Mesdc2 | NM_023403 // mesoderm development |
| | | | | candiate 2 |
| 1437340_x_at | −2.00 | RIKEN cDNA 2200002K21 gene | 2200002K21Rik | NM_025466 // RIKEN cDNA 2200002K21 |
| 1422710_a_at | −1.99 | calcium channel, voltage-dependent, T type, alpha 1H | Cacna1h | NM_021415 // calcium channel alpha13.2 |
| | | subunit | | subunit |
| 1423415_at | −1.97 | G protein-coupled receptor 83 | Gpr83 | NM_010287 // G protein-coupled receptor |
| | | | | 83 |
| 1427600_at | −1.97 | Mus musculus 9.5 days embryo parthenogenote cDNA, | NA | NA |
| | | RIKEN full-length enriched library, clone: | | |
| | | B130041F17 | | |
| | | product: unknown EST, full insert sequence. | | |
| AFFX- | −1.96 | NA | NA | NA |
| 18SRNAMur/X | | | | |
| 00686_M_at | | | | |
| AFFX- | −1.95 | NA | NA | NA |
| 18SRNAMur/X | | | | |
| 00686_5_at | | | | |
| 1452754_at | −1.95 | RIKEN cDNA 5730592L21 gene | 5730592L21Rik | NM_029720 // RiKEN cDNA 5730592L21 |
| 1448812_at | −1.95 | hippocalcin-like 1 | Hpcal1 | NM_016677 // hippocalcin-like 1 |
| 1452270_s_at | −1.95 | cubilin (intrinsic factor-cobalamin receptor) | Cubn | NA |
| 1418360_at | −1.92 | zinc finger protein 179 | Zfp179 | NM_009548 // zinc finger protein 179 |
| 1424051_at | −1.92 | procollagen, type IV, alpha 2 | Col4a2 | NM_009932 // procollagen, type IV, alpha 2 |
| 1420377_at | −1.89 | sialyltransferase 8 (alpha-2, 8-sialyltransferase) B | Siat8b | NM_009181 // sialyltransferase 8 (alpha-2, |
| | | | | 8-sialyltransferase) B |
| 1426604_at | −1.89 | ribonuclease L (2′,5′-oligoisoadenylate synthetase- | Rnasel | NM_011882 // ribonuclease L (2′,5′- |
| | | dependent) | | oligoisoadenylate synthetase-dependent) |
| 1428055_at | −1.88 | Mus musculus cDNA for MBII-343 snoRNA. | NA | NA |
| 1428074_at | −1.86 | RIKEN cDNA 2310037P21 gene | 2310037P21Rik | NA |
| 1424547_at | −1.85 | carbonic anhydrase 10 | Car10 | NA |
| 1427320_at | −1.85 | coatomer protein complex, subunit gamma 2, | Copg2as2 | XR_000142 // |
| | | antisense 2 | | |
| 1437687_x_at | −1.85 | FK506 binding protein 9 | Fkbp9 | NM_012056 // FK506 binding protein 9 |
| 1438968_x_at | −1.84 | serine protease inhibitor, Kunitz type 2 | Spint2 | NM_011464 // serine protease inhibitor, |
| | | | | Kunitz type 2 |
| 1435526_at | −1.83 | hypothetical protein MGC6357 | MGC6357 | NM_144791 // hypothetical protein |
| | | | | MGC6357 |
| 1424415_s_at | −1.82 | RIKEN cDNA D330035F22 gene | D330035F22Rik | NA |
| 1450905_at | −1.82 | plexin C1 | Plxnc1 | NM_018797 // plexin C1 |
| 1429227_x_at | −1.81 | nucleosome assembly protein 1-like 1 | Nap1l1 | NM_015781 // nucleosome assembly protein |
| | | | | 1-like 1 |
| 1424214_at | −1.80 | RIKEN cDNA 9130213B05 gene | 9130213B05Rik | NM_145562 // RIKEN cDNA 9130213B05 |
| 1418983_at | −1.80 | channel-interacting PDZ domain protein | Cipp | NM_007704 // channel-interacting PDZ |
| | | | | domain protein /// NM_172696 // channel- |
| | | | | interacting PDZ domain protein |
| 1448545_at | −1.78 | syndecan 2 | Sdc2 | NM_008304 // syndecan 2 |
| 1417011_at | −1.78 | syndecan 2 | Sdc2 | NM_008304 // syndecan 2 |
| 1449244_at | −1.77 | cadherin 2 | Cdh2 | NM_007664 // cadherin 2 |
| 1425784_a_at | −1.77 | olfactomedin 1 | Olfm1 | NM_019498 // olfactomedin 1 |
| 1455177_at | −1.77 | Abelson helper integration site | Ahi1 | NM_026203 // AHi1 |
| 1448554_s_at | −1.76 | myosin, heavy polypeptide 7, cardiac muscle, beta | Myh7 | NM_080728 // myosin, heavy polypeptide 7, |
| | | | | cardiac muscle, beta |
| 1418091_at | −1.76 | Tcfcp2-related transcriptional repressor 1 | Crtr1-pending | NM_023755 // transcription repressor |
| | | | | CRTR-1 |
| 1417782_at | −1.76 | RIKEN cDNA 2900019C14 gene | 2900019C14Rik | NM_026058 // RIKEN cDNA 2900019C14 |
| 1419879_s_at | −1.76 | tripartite motif protein 25 | Trim25 | NA |
| 1452107_s_at | −1.75 | nephronectin | Npnt | NM_033525 // nephronectin |
| 1449410_a_at | −1.74 | growth arrest specific 5 | Gas5 | NM_013525 // growth arrest specific 5 |
| 1424949_at | −1.74 | upstream regulatory element binding protein 1 | Ureb1-pending | NA |
| 1427670_a_at | −1.74 | transcription factor 12 | Tcf12 | NM_011544 // transcription factor 12 |
| 1426436_at | −1.73 | RIKEN cDNA 8430420C20 gene | 8430420C20Rik | NM_145586 // promethin |
| 1416997_a_at | −1.73 | huntingtin-associated protein 1 | Hap1 | NM_010404 // huntingtin-associated protein |
| | | | | 1 isoform A /// NM_177981 // huntingtin- |
| | | | | associated protein 1 isoform B |
| 1418164_at | −1.73 | epimorphin | Epim | NM_007941 // epimorphin |
| 1438443_at | −1.72 | zinc finger protein 288 | Zfp288 | NM_019778 // zinc finger protein 288 |
| 1423085_at | −1.72 | ephrin B3 | Efnb3 | NM_007911 // ephrin B3 |
| 1428875_at | −1.71 | RIKEN cDNA 3110027H23 gene | 3110027H23Rik | NM_175193 // RIKEN cDNA 3110027H23 |
| 1425110_at | −1.71 | RIKEN cDNA 6330404A12 gene | 6330404A12Rik | NM_025696 // RIKEN cDNA 6330404A12 |
| 1449799_s_at | −1.70 | RIKEN cDNA 1200008D14 gene | 1200008D14Rik | NM_026163 // RIKEN cDNA 1200008D14 |
| | | | | /// NM_027894 // |
| 1421088_at | −1.70 | glypican 4 | Gpc4 | NM_008150 // glypican 4 |
| 1417272_at | −1.69 | RIKEN cDNA 9130005N14 gene | 9130005N14Rik | NM_026667 // RIKEN cDNA 9130005N14 |
| 1448065_at | −1.69 | UDP-Gal: betaGlcNAc beta 1,4-galactosyltransferase, | B4galt3 | NM_020579 // UDP-Gal: betaGlcNAc beta |
| | | polypeptide 3 | | 1,4-galactosyltransferase, polypeptide 3 |
| 1417996_at | −1.68 | neuroglobin | Ngb | NM_022414 // neuroglobin |
| 1417978_at | −1.68 | RIKEN cDNA 1300018P11 gene | 1300018P11Rik | NM_025829 // RIKEN cDNA 1300018P11 |
| 1424659_at | −1.68 | slit homolog 2 (Drosophila) | Slit2 | NM_178804 // slit homolog 2 |
| 1430195_at | −1.67 | RIKEN cDNA 2810043O03 gene | 2810043O03Rik | NA |
| 1421340_at | −1.67 | mitogen activated protein kinase kinase kinase 5 | Map3k5 | NM_008580 // mitogen activated protein |
| | | | | kinase kinase kinase 5 |
| 1425314_at | −1.67 | monogenic, audiogenic seizure susceptibility 1 | Mass1 | NM_054053 // monogenic, audiogenic |
| | | | | seizure susceptibility 1 |
| 1423802_at | −1.67 | cDNA sequence BC017634 | BC017634 | NM_145621 // similar to vesicle-associated |
| | | | | calmodulin-binding protein |
| 1420476_a_at | −1.67 | nucleosome assembly protein 1-like 1 | Nap1l1 | NM_015781 // nucleosome assembly protein |
| | | | | 1-like 1 |
| 1455491_at | −1.67 | Mus musculus, clone IMAGE: 6741456, mRNA | NA | NA |
| 1422433_s_at | −1.66 | isocitrate dehydrogenase 1 (NADP+), soluble | Idh1 | NM_010497 // isocitrate dehydrogenase 1 |
| | | | | (NADP+), soluble |
| 1451799_at | −1.66 | RIKEN cDNA 2610528H13 gene | 2610528H13Rik | NM_145944 // RIKEN cDNA 2610528H13 |
| 1418257_at | −1.65 | solute carrier family 12, member 7 | Slc12a7 | NM_011390 // solute carrier family 12, |
| | | | | member 7 |
| 1455628_at | −1.64 | RIKEN cDNA 6430543G08 gene | 6430543G08Rik | NA |
| 1427501_at | −1.64 | hypothetical protein 5031409G23 | 5031409G23 | NA |
| 1422064_a_at | −1.63 | zinc finger protein 288 | Zfp288 | NM_019778 // zinc finger protein 288 |
| 1422831_at | −1.63 | fibrillin 2 | Fbn2 | NM_010181 // fibrillin 2 |
| 1428113_at | −1.63 | RIKEN cDNA 4930403J22 gene | 4930403J22Rik | NA |
| 1419638_at | −1.62 | ephrin B2 | Efnb2 | NM_010111 // ephrin B2 |
| 1449630_s_at | −1.62 | RIKEN cDNA B930025N23 gene | B930025N23Rik | NA |
| 1426774_at | −1.62 | expressed sequence AA409132 | AA409132 | NM_172893 // expressed sequence |
| | | | | AA409132 |
| 1423852_at | −1.60 | RIKEN cDNA 9430059P22 gene | 9430059P22Rik | NM_145463 // hypothetical protein |
| | | | | BC024118 |
| 1419470_at | −1.60 | guanine nucleotide binding protein, beta 4 | Gnb4 | NM_013531 // guanine nucleotide-binding |
| | | | | protein, beta-4 subunit |
| 1427123_s_at | −1.60 | coatomer protein complex, subunit gamma 2, | Copg2as2 | XR_000142 // |
| | | antisense 2 | | |
| 1427156_s_at | −1.60 | RIKEN cDNA 1700011I11 gene | 1700011I11Rik | NM_029291 // ASC-1 complex subunit P100 |
| 1448940_at | −1.59 | tripartite motif protein 21 | Trim21 | NM_009277 // 52 kD Ro/SSA autoantigen |
| 1439260_a_at | −1.59 | ectonucleotide pyrophosphatase/phosphodiesterase 3 | Enpp3 | NA |
| 1417959_at | −1.59 | RIKEN cDNA 1110003B01 gene | 1110003B01Rik | NM_026131 // RIKEN cDNA 1110003B01 |
| 1435184_at | −1.58 | RIKEN cDNA B430320C24 gene | B430320C24Rik | NA |
| 1460292_a_at | −1.58 | SWI/SNF related, matrix associated, actin dependent | Smarca1 | NM_053123 // SWI/SNF related, matrix |
| | | regulator of chromatin, subfamily a, member 1 | | associated, actin dependent regulator of |
| | | | | chromatin, subfamily a, member 1 |
| 1416111_at | −1.58 | CD83 antigen | Cd83 | NM_009856 // CD83 antigen |
| 1416874_a_at | −1.58 | RIKEN cDNA 5730511K23 gene | 5730511K23Rik | NM_019458 // RIKEN cDNA 5730511K23 |
| 1455796_x_at | −1.58 | olfactomedin 1 | Olfm1 | NM_019498 // olfactomedin 1 |
| 1449236_at | −1.58 | delta-like 3 (Drosophila) | Dll3 | NM_007866 // delta-like 3 isoform 1 |
| 1425264_s_at | −1.57 | myelin basic protein | Mbp | NM_010777 // myelin basic protein |
| 1434935_at | −1.57 | RIKEN cDNA 5530400K14 gene | 5530400K14Rik | NA |
| 1425111_at | −1.57 | RIKEN cDNA 6330404A12 gene | 6330404A12Rik | NM_025696 // RIKEN cDNA 6330404A12 |
| 1422629_s_at | −1.57 | shroom | Shrm | NM_015756 // shroom |
| 1436921_at | −1.55 | ATPase, Cu++ transporting, alpha polypeptide | Atp7a | NM_009726 // ATPase, Cu++ transporting, |
| | | | | alpha polypeptide |
| 1419254_at | −1.55 | methylenetetrahydrofolate dehydrogenase (NAD+ | Mthfd2 | NM_008638 // methylenetetrahydrofolate |
| | | dependent), methenyltetrahydrofolate cyclohydrolase | | dehydrogenase (NAD+ dependent), |
| | | | | methenyltetrahydrofolate cyclohydrolase |
| 1418774_a_at | −1.55 | ATPase, Cu++ transporting, alpha polypeptide | Atp7a | NM_009726 // ATPase, Cu++ transporting, |
| | | | | alpha polypeptide |
| 1415691_at | −1.55 | discs, large homolog 1 (Drosophila) | Dlgh1 | NM_007862 // discs large homolog 1 |
| 1420342_at | −1.54 | ganglioside-induced differentiation-associated-protein | Gdap10 | NM_010268 // ganglioside-induced |
| | | 10 | | differentiation-associated-protein 10 |
| 1423489_at | −1.54 | monocyte to macrophage differentiation-associated | Mmd | NM_026178 // monocyte to macrophage |
| | | | | differentiation-associated |
| 1423851_a_at | −1.54 | RIKEN cDNA 9430059P22 gene | 9430059P22Rik | NM_145463 // hypothetical protein |
| | | | | BC024118 |
| 1455222_a_at | −1.54 | upstream binding protein 1 | Ubp1 | NM_013699 // upstream binding protein 1 |
| 1427052_at | −1.54 | acetyl-Coenzyme A carboxylase beta | Acacb | NA |
| 1416807_at | −1.54 | ribosomal protein L44 | Rpl44 | NM_019865 // ribosomal protein L44 |
| 1453037_at | −1.53 | RIKEN cDNA 1110002E23 gene | 1110002E23Rik | NM_025365 // RIKEN cDNA 1110002E23 |
| 1417220_at | −1.53 | fumarylacetoacetate hydrolase | Fah | NM_010176 // fumarylacetoacetate |
| | | | | hydrolase |
| 1449315_at | −1.53 | odd Oz/ten-m homolog 3 (Drosophila) | Odz3 | NM_011857 // odd Oz/ten-m homolog 3 |
| 1439244_a_at | −1.53 | hypothetical protein MGC11932 | MGC11932 | NM_144925 // hypothetical protein |
| | | | | MGC11932 |
| 1460324_at | −1.53 | Mus musculus transcribed sequences | NA | NA |
| 1452222_at | −1.53 | utrophin | Utrn | NM_011682 // utrophin |
| 1416301_a_at | −1.53 | early B-cell factor 1 | Ebf1 | NM_007897 // early B-cell factor 1 |
| 1419291_x_at | −1.52 | growth arrest specific 5 | Gas5 | NM_013525 // growth arrest specific 5 |
| 1427795_s_at | −1.52 | Mus musculus, clone IMAGE: 6494162, mRNA | NA | NA |
| 1419655_at | −1.52 | transducin-like enhancer of split 3, homolog of | Tle3 | NM_009389 // transducin-like enhancer |
| | | Drosophila E(spl) | | protein 3 |
| 1454789_x_at | −1.52 | RIKEN cDNA 2610031L17 gene | 2610031L17Rik | NM_133701 // RIKEN cDNA 1190003A07 |
| 1419186_a_at | −1.51 | sialyltransferase 8 (alpha-2, 8-sialyltransferase) D | Siat8d | NM_009183 // sialyltransferase 8 (alpha-2, |
| | | | | 8-sialyltransferase) D |
| 1451501_a_at | −1.51 | growth hormone receptor | Ghr | NM_010284 // growth hormone receptor |
| 1423437_at | −1.51 | glutathione S-transferase, alpha 3 | Gsta3 | NM_010356 // glutathione S-transferase, |
| | | | | alpha 3 |
| 1427254_at | −1.51 | expressed sequence AW610627 | AW610627 | NM_173364 // expressed sequence |
| | | | | AW610627 |
| 1423404_at | −1.51 | RIKEN cDNA 2200002K21 gene | 2200002K21Rik | NM_025466 // RIKEN cDNA 2200002K21 |
| 1460637_s_at | −1.51 | prefoldin 5 | Pfdn5 | NM_020031 // prefoldin 5 |
| 1422834_at | −1.51 | potassium voltage-gated channel, Shal-related family, | Kcnd2 | NM_019697 // potassium voltage-gated |
| | | member 2 | | channel, Shal-related family, member 2 |
| 1424632_a_at | −1.51 | REV3-like, catalytic subunit of DNA polymerase zeta | Rev3l | NM_011264 // REV3-like, catalytic subunit |
| | | RAD54 like (S. cerevisiae) | | of DNA polymerase zeta RAD54 like |
| 1417022_at | −1.51 | solute carrier family 7 (cationic amino acid transporter, | Slc7a3 | NM_007515 // solute carrier family 7 |
| | | y+ system), member 3 | | (cationic amino acid transporter, y+ system), |
| | | | | member 3 |
| 1418004_a_at | −1.50 | RIKEN cDNA 1810009M01 gene | 1810009M01Rik | NM_023056 // LR8 protein |
| 1449106_at | −1.50 | glutathione peroxidase 3 | Gpx3 | NM_008161 // glutathione peroxidase 3 |
| 1448925_at | −1.50 | twist homolog 2 (Drosophila) | Twist2 | NM_007855 // twist homolog 2 |
| 1438802_at | −1.50 | forkhead box P1 | Foxp1 | NM_053202 // forkhead box P1 |
| 1419967_at | −1.50 | sec13-like protein | Sec13l-pending | NM_028112 // sec13-like protein |

Other Embodiments

All publications and patent applications cited in this specification are herein incorporated by reference as if each individual publication or patent application were specifically and individually indicated to be incorporated by reference. Although the foregoing invention has been described in some detail by way of illustration and example for-purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of identifying a compound for treating or preventing Parkinson's disease, said method comprising the steps of:
(a) providing cells that express at least five genes selected from Table 4;
(b) contacting said cells with a candidate compound; and
(c) assessing the expression level of said genes relative to the expression level of said genes in the absence of said candidate compound, wherein a candidate compound that reduces the expression of at least three of said genes is identified as a compound useful for treating or preventing Parkinson's disease.

2. The method of claim 1, wherein said cells comprise more than one cell-type.

3. The method of claim 1, wherein all of said genes are expressed in a single cell-type.

4. The method of claim 1, wherein said cells express at least 10 genes selected from Table 4.

5. The method of claim 1, wherein said cells express at least 50 genes selected from Table 4.

6. The method of claim 2, wherein said each cell-type is segregated from the other cell-types during steps (b) and (c).

7. The method of claim 1, wherein said contacting further comprises contacting said cell with a mitochondrial complex I inhibitor.

8. The method of claim 7, wherein said mitochondrial complex I inhibitor is 1-methyl-4-phenylpyridinium (MPP⁺), rotenone, isoquinoline, tetrahydroisoquinoline, or 6-hydroxydopamine.

9. The method of claim 1, wherein said cells are mammalian cells.

10. The method of claim 9, wherein said cell is a human cell or a rodent cell.

11. The method of claim 10, wherein at least one of said cells is a dopaminergic cell or a cell isolated from the ventral mesencephalic tissue.

12. The method of claim 1, wherein at least one of said cells is a PC12 cell.

13. The method of claim 12, wherein said PC12 cells overexpress α-synuclein.

14. The method of claim 1, wherein said assessing step (c) comprises measuring the RNA levels transcribed from said gene.

15. The method of claim 1, wherein said assessing step (c) comprises measuring the amount of said protein encoded by said gene.

16. The method of claim 1, wherein at least one of said genes is recombinantly expressed by said cells.

17. A method of identifying a compound for treating or preventing Parkinson's disease, said method comprising the steps of:
(a) providing cells that expresses at least five genes selected from Table 5;
(b) contacting said cells with a candidate compound; and
(c) assessing the expression level of said genes relative to the expression level of said protein in the absence of said candidate compound, wherein a candidate compound that increases the expression of at least three of said genes is identified as a compound useful for treating or preventing Parkinson's disease.

18. The method of claim 17, wherein said cells comprise more than one cell-type.

19. The method of claim 17, wherein all of said genes are expressed in a single cell-type.

20. The method of claim 17, wherein said cells express at least 10 genes selected from Table 5.

21. The method of claim 17, wherein said cells express at least 50 genes selected from Table 5.

22. The method of claim 18, wherein said each cell-type is segregated from the other cell-types during steps (b) and (c).

23. The method of claim 17, wherein said contacting further comprises contacting said cell with a mitochondrial complex I inhibitor.

24. The method of claim 23, wherein said mitochondrial complex I inhibitor is 1-methyl-4-phenylpyridinium (MPP⁺), rotenone, isoquinoline, tetrahydroisoquinoline, or 6-hydroxydopamine.

25. The method of claim 17, wherein said cells are mammalian cells.

26. The method of claim 25, wherein said cell is a human cell or a rodent cell.

27. The method of claim 26, wherein at least one of said cells is a dopaminergic cell or a cell isolated from the ventral mesencephalic tissue.

28. The method of claim 17, wherein at least one of said cells is a PC12 cell.

29. The method of claim 28, wherein said PC12 cells overexpress α-synuclein.

30. The method of claim 17, wherein said assessing step (c) comprises measuring the RNA levels transcribed from said gene.

31. The method of claim 17, wherein said assessing step (c) comprises measuring the amount of said protein encoded by said gene.

32. The method of claim 17, wherein at least one of said genes is recombinantly expressed by said cells.

33. A method of identifying a compound for treating or preventing Parkinson's disease, said method comprising the steps of:
(a) providing a cell comprising a reporter gene operably linked to the promoter of a gene selected from Table 4;
(b) contacting said cell with a candidate compound; and
(c) assessing the level of expression of said reporter gene relative to the level of expression of said reporter gene in the absence of said candidate compound, wherein a candidate compound that reduces the level of expression of said reporter gene is identified as a compound that is useful for the treatment or prevention of Parkinson's disease.

34. The method of claim 33, wherein said reporter gene is selected from the group consisting of glucuronidase (GUS), luciferase, chloramphenicol transacetylase (CAT), green fluorescent protein (GFP), alkaline phosphatase, and β-galactosidase.

35. The method of claim 33, wherein said contacting further comprises contacting said cell with a mitochondrial complex I inhibitor.

36. The method of claim 35, wherein said mitochondrial complex I inhibitor is 1-methyl-4-phenylpyridinium (MPP⁺), rotenone, isoquinoline, or tetrahydroisoquinoline.

37. The method of claim 36, wherein said cell is a human cell or a rodent cell.

38. The method of claim 33, wherein said cell is a dopaminergic cell or a cell isolated from the ventral mesencephalic tissue.

39. The method of claim 33, wherein said cell is a PC12 cell.

40. The method of claim 39, wherein said PC12 cell overexpresses α-synuclein.

41. A method of identifying a compound for treating or preventing Parkinson's disease, said method comprising the steps of:
(a) providing a cell comprising a reporter gene operably linked to the promoter of a gene selected from Table 5;
(b) contacting said cell with a candidate compound; and
(c) assessing the level of expression of said reporter gene relative to the level of expression of said reporter gene in the absence of said candidate compound, wherein a candidate compound that increases the level of expression of said reporter gene is identified as a compound that is useful for the treatment or prevention of Parkinson's disease.

42. The method of claim 41, wherein said reporter gene is selected from the group consisting of glucuronidase (GUS), luciferase, chloramphenicol transacetylase (CAT), green fluorescent protein (GFP), alkaline phosphatase, and β-galactosidase.

43. The method of claim 41, wherein said contacting further comprises contacting said cell with a mitochondrial complex I inhibitor.

44. The method of claim 43, wherein said mitochondrial complex I inhibitor is 1-methyl-4-phenylpyridinium (MPP⁺), rotenone, isoquinoline, or tetrahydroisoquinoline.

45. The method of claim 41, wherein said cell is a mammalian cell.

46. The method of claim 45, wherein said cell is a human cell or a rodent cell.

47. The method of claim 41, wherein said cell is a dopaminergic cell or a cell isolated from the ventral mesencephalic tissue.

48. The method of claim 41, wherein said cell is a PC12 cell.

49. The method of claim 48, wherein said PC12 cell overexpresses α-synuclein.

50. A solid support surface comprising 1000 or fewer unique polynucleotide probes capable of binding at least 200 distinct nucleic acids that encode the genes of Table 4 and Table 5, wherein said probes are arranged on said surface such that, when contacted with a sample containing said nucleic acids, each binding event is segregated from the others.

51. The solid support surface of claim 50, wherein said 1000 or fewer unique polynucleotide probes capable of binding at least 300 distinct nucleic acids that encode the genes of Table 4 and Table 5.

52. The solid support surface of claim 50, wherein said 1000 or fewer distinct polynucleotide probes capable of binding at least 400 distinct nucleic acids that encode the genes of Table 4 and Table 5.

53. The solid support surface of claim 50, wherein said polynucleotide probes are conjugated to a detectable label.

54. The solid support surface of claim 53, wherein said detectable label is a fluorescent label or an enzyme tag.

55. The solid support surface of claim 54, wherein said enzyme tag is selected from the group consisting of digoxigenin, β-galactosidase, urease, alkaline phosphatase, peroxidase, or avidin/biotin complex.

56. The surface of claim 55, wherein said surface is a multiwell plate.

57. The surface of claim 55, wherein said surface is a slide.
